(19) United States
(12) Patent Application Publication
(10) Pub. No.: US 2004/0126835 A1
(43) Pub. Date: Jul. 1, 2004

(54) Cell assays

(76) Inventors: Ann Burchell, Dundee (GB); Robert Hume, Dundee (GB)

Correspondence Address: Karla M Weyand, Rogalskyj & Weyand, P O Box 44, Livonia, NY, 14487-0044, US

(21) Appl. No.: 10/363,767

(22) PCT Filed: Sep. 7, 2001
(86) PCT No.: PCT/GB01/04010

(30) Foreign Application Priority Data
Sep. 8, 2000 (GB) 0022017.8

(51) Int. Cl. C12Q 1/02; C12Q 1/04

(52) U.S. Cl. 435/29; 435/34

(57) ABSTRACT

A method for identifying and/or characterising a target cell in, and/or isolating a target cell from, a test sample which may contain said target cell, comprising the steps of 1) identifying and/or characterising any target cell in, and/or isolating any target cell from, the test sample by performing on the test sample a process involving interaction of a target cell selective reagent with a characterising component of the target cell, 2) providing a positive control sample comprising a positive control cell, wherein the positive control cell comprises said characterising component and is not identical to the target cell, 3) identifying and/or characterising the positive control cell in, and/or isolating the positive control cell from, the positive control sample by a process according to step 1 performed on the positive control sample, wherein the method for identifying, isolating and/or characterising the target cell is considered to have been performed correctly if the positive control cell is identified, isolated and/or characterised correctly in step 3. Recombinant control cells may be particularly useful, for example in relation to methods of isolating and analysing fetal cells, fore example fetal cells obtained from maternal blood.

The present invention relates to assays for the identification, isolation and/or characterisation of cells in a sample.

There are numerous situations in which it is desirable to be able to identify, isolate and/or characterise types (and numbers) of cells present in a sample. For example, it may be desirable to detect and/or quantitate the presence of bacteria or malignant cells in a pathological specimen, and to characterise further the bacteria or malignant cells, for example in order to determine whether the cells are resistant to particular drugs.

Methods of bacterial identification in current clinical use involve preparing cultures from the test sample and are dependent on chemical staining patterns, coupled with culture specific characteristics. This traditional process is by its very nature slow, dependent on growth of sufficient quantities of the organism, time consuming, and costly in terms of staff time. The time delay in obtaining test results from such processes can adversely affect the prognosis for the patient. For example, it may take 48 hours to obtain a clinically useful result from a sample from a sick child with a potentially life threatening disease. Meanwhile, the child is treated on the basis of best therapeutic guess based upon clinical experience. Present diagnostic tests are therefore of limited use in directing treatment of acute patients. This situation is unsatisfactory for clinicians and patients.

In addition, this process of identification is dependent on the skill of the operator in interpreting the results of, for example, culturing experiments, and therefore requires experienced staff. Essential clinical information such as antibiotic resistance patterns of the organism is only available after clinically significant delay and interpretation of the tests used in the laboratory setting is semiquantitative at best. Controls are not routinely used.

In addition, natural control samples which could be used in the diagnostic tests may be infectious, requiring category 3 containment facilities. Therefore growing stocks of these potentially dangerous organisms for control purposes is not desirable and transporting between laboratories potentially hazardous to public safety.

Methods for performing bacterial diagnosis by detection/isolation of small numbers from a relatively large volume of bodily fluid are not used routinely in the clinic. PCR-based methods, as described in various patent applications, for example U.S. Pat. No. 5,994,066, are generally not used in the clinic in routine identification of potentially infective organisms. This may be because of an unacceptably high incidence of false positive and/or false negative results. The methods are also expensive and any cost savings from improvements in the speed and/or accuracy of diagnosis in reducing other healthcare costs has not been calculated, which may also have contributed to use of the methods not having become established routine practice. Current diagnostic procedures are limited at present by the need to increase numbers of organisms prior to diagnosis. As noted above, this entails delays in obtaining the test results.

Controls for slide based diagnostic technology (or other automatable technology), particularly in the clinic, have been heavily reliant on the serendipitous acquisition of a previously positive natural specimen, which can then be compared to the test sample. In addition, these positive specimens may be limited in quantity, availability and specificity. Further, particular positive controls may be specific to individual institutions, making it difficult to apply quality control systems of the standard required for other diagnostic systems. These natural control samples have often been collected in the past under limited ethical constraints and may not be considered ethical today; for example, use of a section from a human tumour may no longer be considered appropriate. The use of tissue for research, teaching, quality control or commercial purposes is constrained by a recent series of recommendations by the Royal College of Pathologists, which are contained in the following documents: The consensus statement of recommended policies for uses of human tissue in research, education and quality control (1999); Examination of the body after death: Information about post-mortem examinations for relatives (March 2000); and Guidelines for the retention of tissues and organs at post-mortem examination (March 2000). There may also be safety (for example infectivity) concerns with such samples.

Similarly, there are major ethical and legal constraints on the use of human fetal tissue, for example as controls in slide based diagnosis of disease (or other automatable technology). The Review of the Guidance on the Research Use of Fetuses and Fetal Material HMSO 1989 (Polkinghorne Report) discusses these constraints in the UK. In particular, it indicates that projects using fetal tissue for the development of diagnostic procedures should be subject to constant ethical reviews and no profit should be made from any transactions involving the disposal of any contents of the uterus. There are similar constraints in the USA: Withdrawal Of Interim NIH Guidelines For The Support And Conduct Of Therapeutic Human Fetal Tissue Transplantation Research In Light Of Superseding Provisions Of Public Law 103-43, The National Institutes Of Health Revitalization Act Of 1993 NIH GUIDE, Volume 22, Number 32, Sep. 3, 1993. It is similarly generally considered to be unethical to grow and use cells recovered from human embryos as a result of in-vitro fertilisation for this purpose (Human Fertilisation and Embryology Act UK 1990).

The difficulties with current assay techniques are well known. There are many patents and patent applications concerning possible improvements in diagnostic assays, for example using PCR-based techniques with purified nucleic acid as a control, or antibody-based techniques using purified polypeptides as a control. Examples include WO99/09186; U.S. Pat. No. 6,008,201; U.S. Pat. No. 5,871,912; U.S. Pat. No. 5,851,761; WO98/02547; U.S. Pat. No. 5,916,558; WO98/04711; U.S. Pat. No. 5,788,962; WO99/24577; WO98/53076; WO98/36089; U.S. Pat. No. 5,919,640; U.S. Pat. No. 5,994,066; U.S. Pat. No. 6,013,514; U.S. Pat. No. 6,001,564; WO98/53102; U.S. Pat. No. 5,827,651; U.S. Pat. No. 5,770,719. None suggest the methods of the present invention.

The present invention concerns control cells for use in methods for isolating cells from, and/or identifying and/or characterising cells in, a test sample. The control cells are cells, typically genetically engineered cells, which are abundant and safe, which act as positive or negative controls in diagnostic assays which involve the identification, characterisation or isolation of a cell in a test sample. It will be appreciated that such methods may further provide information on the abundance of the isolated/identified/characterised cells, ie provide quantitative information. Provision of control cells allows assays to be performed that were not previously attempted in a routine manner, for example assays for small numbers of target cells in a biologically complex or relatively large volume sample. It was not previously realised that such assays could be feasible, nor what developments were required in order to render the assays feasible. It was not previously realised that the use of control cells provided by the present invention is desirable.

The present invention provides the use of control cells in a wide range of diagnostic and/or isolation techniques, which only become feasible with the recognition of the need for, and provision of, the relevant control cells. Control cells may be used in the development of new diagnostic and/or isolation techniques etc which may themselves need the use of control cells in routine operation. Control cells may also be used as part of existing assay methods, leading to potential improvements in accuracy, sensitivity, selectivity, quantitative range and/or speed of diagnosis, and potential improvement of clinical value.

There have been no suggestions that control cells are useful in the development of new diagnostic methods. This is particularly so for the potential development of diagnostic methods using individual cells. Currently, single cell diagnosis is not carried out for the reason that the identity, and pathogenicity of the organism or the site of origin of the cell cannot be determined reliably, because of the difficulties of providing controls. The present invention further provides the use of control cells in relation to single cell diagnosis and other investigations of single cells.

In summary, the present invention provides methods for isolating and/or analysing bacterial cells or other microorganism cells in which control cells are used. Typically, the bacterial or other microorganism cells under investigation are pathogenic or potentially pathogenic. Methods for isolating and/or analysing higher eukaryotic cells, for example abnormal cells, for example cancerous or infected host cells, in which control cells are used, are also provided. The methods are preferably slide-based methods, or other methods suitable for high-throughput operation.

In particular, the present invention provides methods for isolating and/or analysing embryonic and fetal cells from maternal circulation in which control cells are used. The methods preferably make use of the methods for isolating or identifying embryonic and fetal cells described in WO98/40746.

A first aspect of the invention provides a method for identifying and/or characterising a target cell in, and/or isolating a target cell from, a test sample which may contain said target cell, the method comprising the steps of

(1) identifying and/or characterising any target cell in, and/or isolating any target cell from, the test sample by performing on the sample a process involving interaction of a target cell selective reagent with a characterising component of the target cell,

(2) providing a positive control sample comprising a positive control cell, wherein the control cell comprises said characterising component and is not identical to the target cell,

(3) identifying and/or characterising the positive control cell in, and/or isolating the positive control cell from, the positive control sample by a process according to step 1 performed on the positive control sample, wherein the method for identifying, isolating and/or characterising the target cell is considered to have been performed correctly if the positive control cell is identified, isolated and/or characterised correctly in step 3.

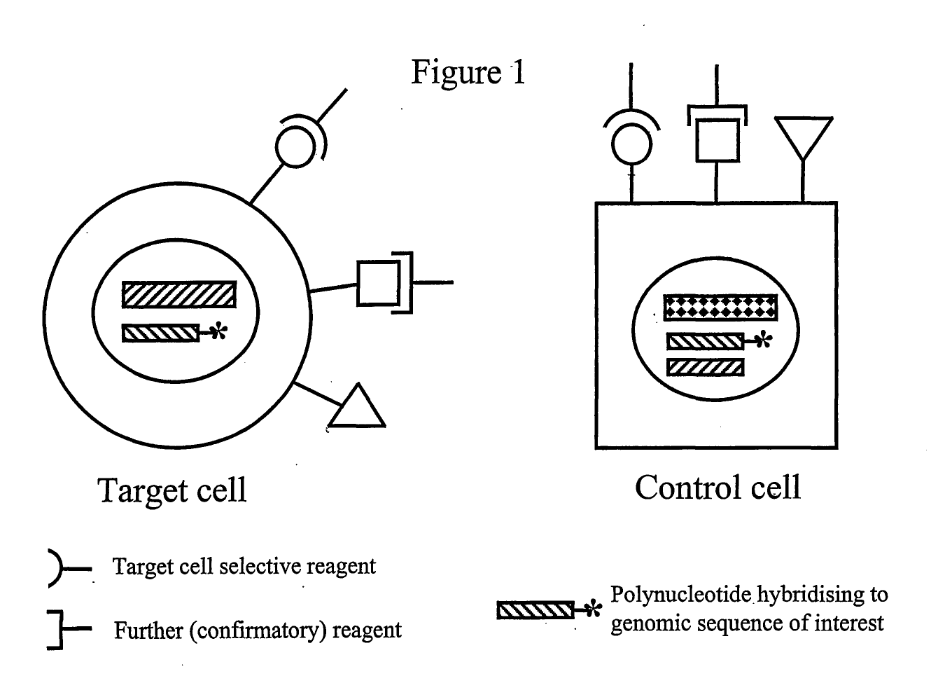

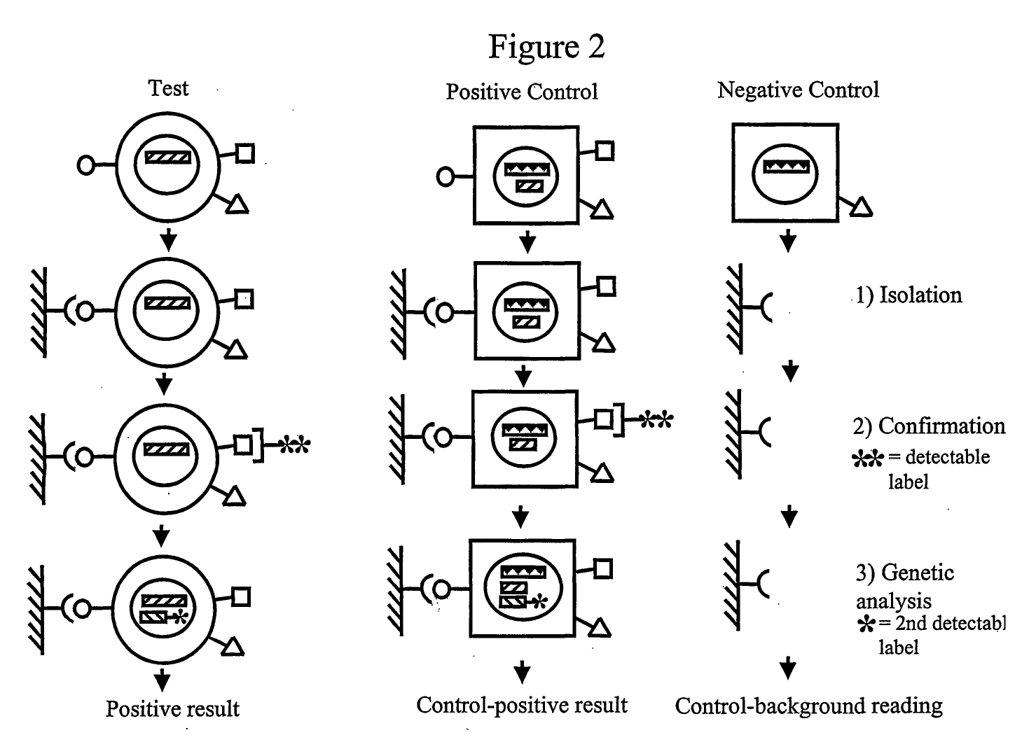

FIG. 1 shows a diagrammatic representation of properties of the control cell compared to the target cell in this aspect of the invention and in particular embodiments of the invention as discussed in more detail below. FIG. 2 shows a diagrammatic representation of how such a control cell and related negative control cell may be used in a method of this aspect of the invention and in particular embodiments of the invention as discused in more detail below.

It is preferred in relation to all aspects of the invention that the positive control cell is recombinant. However, it may also be a non-identical cell which comprises the characterising component (but is not expected to be present in the test sample and hence is not a target cell). For example, a liver cell may be used (though is not preferred for reasons of convenience) as a positive control cell in relation to fetal cell isolation, as discussed further below.

A second aspect of the invention provides a method for determining whether a method for identifying and/or characterising a target cell in, and/or isolating a target cell from, a test sample which may contain said target cell has been performed correctly, comprising the steps of

(1) identifying and/or characterising any target cell in, and/or isolating any target cell from, the test sample by performing on the test sample a process involving interaction of a target cell selective reagent with a characterising component of the target cell,

(2) providing a positive control sample comprising a positive control cell, wherein the positive control cell comprises said characterising component and is not identical to the target cell,

(3) identifying and/or characterising the positive control cell in, and/or isolating the positive control cell from, the positive control sample by a process according to step 1 performed on the positive control sample,

(4) determining whether the positive control cell is identified, isolated and/or characterised correctly in step 3.

A third aspect of the invention provides the use of a positive control cell in a method for identifying and/or characterising a target cell in, and/or isolating a target cell from, a test sample which may contain said target cell, wherein any target cell in the test sample is identified, isolated and/or characterised by a process involving interaction of a test cell selective reagent with a characterising component of the target cell, wherein the positive control cell comprises said characterising component and is not identical to the target cell. The positive control cell is used in assessing and controlling the quality of performance of the method ie in determining whether it has been performed correctly, and in assessing the reliability of the method.

By “identified, isolated and/or characterised correctly” is meant that the expected identification, isolation or characterisation has been achieved when the method is performed. Thus, if the positive control cell is identified, isolated and/or characterised as expected, the method is deemed to have been performed correctly. The results obtained on the test sample may then be treated as reliable. If the positive control cell is not identified, isolated and/or characterised as expected, then the result obtained on the test sample cannot be relied upon.

It will be appreciated that the results of carrying out the method may be expressed as a positive/negative determination or as a quantitative determination. If only a positive/negative determination is required, then a positive result with one (or more) positive control sample(s) may be sufficient to conclude that the method has been performed correctly. If a quantiative determination is required, it may be necessary to construct a calibration curve/equation, as will be well known to those skilled in the art, using the positive control cells in order to determine that the required sensitivity is being achieved. The dose response determination may further be used in interpreting the results obtained from the test samples on the basis of the calibration curve/equation.

Negative control cells may additionally be used in an analogous way to the (positive) control cells indicated above, for example in order to determine whether the required selectivity is being achieved and/or to assist in distinguishing a positive signal from background. Such negative control cells do not comprise the characterising component (or further components, as discussed below). They may preferably be the parent cell type from which the positive control cell is derived, as discussed further below. Thus, it is preferred that the negative control cell differs from the positive control cell only in relation to the characterising component (and optionally any further components, as discussed below) and any components necessary for the control cell to comprise the characterising (or further) component, for example any polynucleotide sequence encoding the characterising component.

Thus, in a preferred embodiment of a method according to the first aspect of the invention, the method further comprises the steps of (4) providing a negative control sample comprising a negative control cell but not a target cell or positive control cell, wherein the negative control cell does not comprise said characterising component; (5) performing the process according to step 1 on the negative control sample; and (6) determining whether any cell is isolated from, and/or identified and/or characterised in, the negative control sample; or determining the background (negative) result of the process according to step 1. The method for identifying, isolating and/or characterising the target cell is considered to have been performed correctly if the positive control cell is identified, isolated and/or characterised correctly in step (3) and no cell is identified, isolated or characterised in step (6) or if the positive control cell is identified, isolated and/or characterised correctly in step (3) having regard to the background result determined in step (6).

In a preferred embodiment of a method according to the second aspect of the invention, the method further comprises the steps of providing a negative control sample comprising a negative control cell but not a target cell or positive control cell, wherein the negative control cell does not comprise said characterising component; performing the process according to step 1 on the negative control sample; and determining whether any cell is isolated from, and/or identified and/or characterised in, the negative control sample; or determining the background (negative) result of the process according to step 1. If the background result is determined, then in step (4), it is determined whether the positive control cell is identified, isolated and/or characterised correctly in step (3) having regard to the background result.

In the method of the first or second aspect of the invention or use of the third aspect of the invention the step of identifying and/or characterising any target cell in, and/or isolating any target cell from, the test sample may further comprise the step of performing on the test sample or on any target cell isolated from said test sample a process involving selective interaction of one or more further reagents with one or more further components of the target cell. The positive control cell preferably further comprises said further component(s).

Examples of suitable further components and further reagents are described below. Typically, one further reagent/component may allow confirmation that the correct cell type has been identified or isolated, and a still further reagent/component may allow characterisation of the identified or isolated cell, for example in terms of the presence of a particular mutation associated with disease, or as belonging to a particular strain of bacteria or being resistant to particular antibiotics.

For example, in relation to a method of isolating fetal cells from maternal blood in order to test for the presence or absence of a genetic defect such as the gene linked with cystic fibrosis, a positive control cell may be a non-human mammalian cell engineered to express (1) a fetal cell component that is to be used as the basis for isolating the fetal cells, for example a cell surface human adult liver component (discussed further below), (2) a further fetal cell component which can be used to confirm that the isolated cells are fetal cells, for example an intracellular human adult liver component, and (3) the part of the gene linked with cystic fibrosis that is to be tested for, for example using PCR or in situ hybridisation, in the fetal cells.

A further aspect of the invention provides a kit of parts suitable for performing a method for identifying and/or characterising a target cell in, and/or isolating a target cell from, a test sample which may contain said target cell, comprising

1) a target cell selective reagent suitable for interacting with a characterising component of the target cell and

2) a positive control cell, wherein the positive control cell comprises said characterising component and is not identical to the target cell.

The kit preferably further comprises a negative control cell, wherein the negative control cell does not comprise the characterising component. As noted above, the positive control cell is preferably recombinant. It is further preferred that the positive and negative control cells differ substantially only in relation to the presence/absence of the characterising component (including a component necessary for its presence in the cell, for example a recombinant polynucleotide encoding the component).

Such a kit of parts may be suitable for performing a method according to the first or second aspects of the invention.

The kit of parts may further comprise one or more further reagents capable of interacting selectively with one or more further components of the target cell. Preferably the positive control cell further comprises said further component(s). Preferably the negative control cell does not comprise such further component(s).

It will be appreciated that more than one positive control cell (and more than one negative control cell) may be used when analysing a test sample. For example, the test sample may be analysed using more than one target cell selective reagent and a positive control cell (and preferably a negative control cell) may be provided in relation to each such target cell selective reagent.

Whilst the construction of control cells preferably involves living cells, the control cell may be living or dead during its use. Thus, the kit may, for example, comprise dead positive and negative control cells. The control cells may conveniently be immobilised on a solid support, for example in a format suitable for slide-based automated analysis. Thus, an array of positive (and optionally negative) control cells (for example sharing a characterising component with different bacterial strains) may be formed on a solid support, for example a microscope slide using array-forming techniques well known to those skilled in the art, including single-cell immobilisation techniques, as discussed further below.

Thus, a kit of parts as described above may conveniently comprise

1) a target cell selective reagent suitable for interacting with a characterising component of the target cell and

2) a solid support suitable for use in a method for identifying and/or characterising a target cell in, and/or isolating a target cell from, a test sample, on which is immobilised a positive control cell, wherein the positive control cell comprises said characterising component and is not identical to the target cell. Preferably a negative control cell, wherein the negative control cell does not comprise the characterising component, is also immobilised on the solid support. Still more preferably an array of different positive and negative control cells, each positive control cell relating to a different target cell selective reagent, is immobilised on the solid support. It will be appreciated that a single type of negative control cell may be suitable as a negative control in relation to several different positive control cells, for example when the different positive control cells are derived from the same parent cell type. Thus, it may not be necessary to include a separate negative control cell sample in relation to each positive control cell type. As noted above, the solid support is preferably suitable for use in slide-based or other automated analysis. It is preferred that the solid support transmits light with minimal distortion, absorption and emission (ie may be considered to be of optical grade, as known to those skilled in the art); has dimensions consistent with use with a microscope or other device to monitor the absorption, emission or transmission of electromagnetic radiation, preferably visible wavelength light. Thus, the solid support may conveniently be a microscope slide, preferably a glass microscope slide, of typical dimensions 25 mm×75 mm×1 mm, as well known to those skilled in the art. It is preferred that the solid support is impervious to the fixative to be used (if any), preferably impervious to common fixatives, and non-reactive to the diagnostic reagents. Cells may be attached to the support without using a fixative; for example, bacteria may be attached to glass slides without fixative by heat coagulation. Similarly, antibodies may be attached to plastic, for example in multi-well plates, by evaporation.

The solid support may be rigid (for example glass) but it may be flexible, for example a thin, flexible plastic-like material, such as Clingfilm. It may alternatively be liquid, so long as the spatial arrangement of the cells can be controlled. Thus, the cells may be suspended at the interface between two liquids (preferably having the optical and chemical properties indicated above) of different densities.

A further aspect of the invention provides a solid support suitable for use in a method for identifying and/or characterising a target cell in, and/or isolating a target cell from, a test sample, on which is immobilised 1) a positive control cell, wherein the positive control cell comprises a characterising component and 2) a negative control cell, wherein the negative control cell does not comprise the characterising component. It is preferred that the positive and negative control cells differ substantially only in relation to the presence/absence of the characterising component (including a component necessary for its presence in the cell, for example a recombinant polynucleotide encoding the component). The characterising component of the positive control cell is a component which is also present in the intended target cell. The positive control cell is not identical to the intended target cell. Preferences in relation to the solid support and the immobilisation of control cells thereon are as discussed in relation to the kit of parts. Preferred control cells include cells of the invention, as discussed further below and the corresponding negative control cells (parent cells). Preferences for the solid support are as indicated above.

Properties of the Characterising Component and Further Components of the Target Cell

The choice of characterising component will be determined by the definition of the target cell which is to be isolated, identified and/or characterised. The choice of any further component(s) (target cell) will also be determined by the definition of the target cell and the level to which it is required to characterise the target cell.

For example, when seeking to determine how to treat a patient with a suspected bacterial infection, it may first be necessary to determine whether bacteria are present in an appropriate sample from the patient in which bacteria would not normally be expected to be present in significant numbers, for example in blood. Thus, the target cell definition is “any bacterial cell” and the characterising component is a component present in all bacterial cells but not present in eukaryotic cells, for example the patient's cells. The target cell selective reagent is in this case a reagent which interacts with the component present in all bacterial cells, and the positive control cell comprises the same component. A negative control cell (the use of which is desirable) does not comprise this component. The positive and negative control cells may be, for example, yeast cells.

In a different situation, it may be desired to determine whether a particular type of bacterium is present in a sample in which other bacteria are normally present, for example a sample from the mouth. In this case, the target cell is the particular type of bacterium and the characterising component (with which the target cell selective reagent interacts) is a component found in that type of bacteria but not in other bacteria. In this case, the positive and any negative control cells may be bacterial cells of a different type to the target cell type; the positive control cell comprises the characterising component of the target cell type, and the negative control cell does not.

It is preferred that the characterising component is a cell surface component. However, it is not essential that the characterising component is a cell surface component, particularly if the method is for identifying (rather than isolating) the target cell. If the method is for isolating the target cell, it is strongly preferred that the characterising component is a cell surface component. It is further preferred that the target cell selective reagent binds to a portion of the characterising component that is exposed on the surface of the cell. The cell surface component may be, for example, a transmembrane protein, cell wall constituent or periplasmic protein, depending upon the cell type.

Although it is preferred that the characterising component is a cell surface component in relation to isolating the target cell, methods of isolating cells on the basis of intracellular components are know, for example fluorescence-activated cell sorting (FACS) on the basis of a cell-permeable reagent (including a reagent that is capable of entering a permeabilised cell) which is capable of binding to the intracellular component and is capable of being fluorescently labelled. If such a method is used, for example, the characterising component need not be a cell surface component. For example, the characterising component may be a polynucleotide or portion of a polynucleotide; and the target cell selective reagent may be a cell-permeable reagent capable of selectively hybridising to that portion of the polynucleotide. The positive control cell in such a case comprises a polynucleotide comprising at least the portion of the polynucleotide that is capable of hybridising selectively to the cell-permeable reagent. A negative control cell in such a case does not comprise a polynucleotide that is capable of hybridising selectively to the cell-permeable reagent.

As noted above, the control cell may further comprise other components (which may be recombinant) which are also present in the target cell. For example, it may comprise a second component which is also characteristic of the target cell. This may be a component present in the same cell types as the first component (for example in all bacterial cells), or it may be a component present in a narrower class of cell types than the first component (for example, in relation to the above example, only in gram-negative bacteria).

An optional further component of the target cell (and optionally of the is control cell) as indicated above may be a polynucleotide or a polypeptide. As noted above, it may be desirable to make use of more than one further component. Typically, at least one such further component is a polynucleotide, for example a polynucleotide associated with a genetic condition or disease. The condition may be, for example, cancer, or multi-drug resistance.

It will be appreciated that the characterising component (or further component) may be a portion of a molecule present in the target cell. For example, the target cell selective reagent may interact with a portion (characterising component) of a polypeptide present in the target cell. It is not necessary that the positive control cell comprises the full length polypeptide; it may be sufficient for only the portion (characterising component) of the polypeptide that interacts with the target cell selective reagent to be present in the positive control cell, so long as the target cell selective reagent is still able to interact with the characterising component as presented in the positive control cell in substantially the same manner (for example with substantially the same affinity and specificity, or under the same assay conditions) as with the characterising component as presented in the target cell. The characterising component (or other component) may be, for example, a lipid, carbohydrate, glycoprotein or lipoprotein. It is preferred that if the characterising component (or other component) is a polypeptide (by which is included glycoprotein or lipoprotein), then it is present in the positive control cell in the form of a polypeptide with the same sequence as the polypeptide of which it forms part in the target cell.

In those cases where it is desirable to express a component on a cell surface in a cell types that is different, or it is preferable to express only a portion of a cell surface component, for example the most divergent portion of an isoform, then it may be necessary or desirable to construct a vector with a whole or portion of a protein that is normally expressed on the cell surface to which the component of interest is attached. This is feasible, as known to those skilled in the art; for example, green fluorescent protein is often attached to other proteins, including cell surface proteins, that retain their normal sub-cellular distribution pattern.

It will be appreciated that it is not essential that the characterising component (or further component) present in the positive control cell has an identical polynucleotide or polypeptide sequence (as appropriate) to the characterising component (or further component) present in the target cell, so long as the characterising component present in the positive control cell is still able to interact with the target cell selective reagent (or other reagent, as appropriate) in substantially the same manner (for example with substantially the same affinity and specificity, or under the same assay conditions) as with the characterising component present in the target cell.

Permissible modifications, for example particular substitutions, insertions, deletions or fusions, may readily be determined for a particular reagent/component pair, using techniques of measuring intermolecular interactions well known to those skilled in the art. Examples of such techniques include co-immunoprecipitation or surface plasmon resonance methods, the yeast two-hybrid system for detecting interactions, or copurification methods. Techniques for measuring hybridisation between polynucleotides also include surface plasmon resonance methods and other methods well known to those skilled in the art. Bohling et al (1999) Lab Invest 79(3), 337-345, for example, describes a method for detecting the bcl-1/JH translocation in mantle cell lymphoma in which SYBR Green and fluorescence melting curve analysis is used. Such analysis may be carried out, for example, using a machine such as the DASH™ system from Hybaid. The fluorescence from a double-strand nucleic acid-specific dye is measured along a temperature gradient, allowing the T_{mS} of the double-stranded nucleic acids in the sample to be determined.

Properties of the Target Cell Selective Reagent and Further Reagents

As noted above, the isolation/identification/characterisation method involves a target cell selective reagent interacting with a characterising component of the target cell.

By “interacts with” is included the meaning that the cellular component is capable of binding to the reagent. Preferably, the component and reagent are capable of binding with high affinity and specificity for each other. Thus, it is preferred that the component and reagent are specific binding partners. For example, the component and reagent may be complementary polynucleotides that are capable of hybridising to each other under given conditions. Alternatively, the component and reagent may be an antigen and antibody that are capable of binding to each other.

By “high affinity” is meant an interaction with a K_{d} of between 10⁻¹³ and 10⁻¹⁶ M. By “interacts specifically” is meant that the component interacts with at least 100-fold higher affinity (and preferably at least 500-fold, or at least 1000-fold, or at least 2000-fold higher affinity) with the relevant reagent than with other molecules that may be encountered by either the said component or reagent when performing the method.

The reagent may be a substrate of the component (or, less preferably, vice versa). It is preferred that the component acts on such a reagent to convert it to or from a detectable substance (for example, to or from a fluorescent substance). For example, the component may be glucose-6-phosphatase, for which histochemical methods are available. Thus, the substrate may be glucose-6-phosphate, and the colour in the histochemical stain may be produced by a secondary reaction which reveals the presence of the product phosphate.

It will be appreciated that it may not be necessary for the component and reagent to interact with high affinity, as defined above; for example, the requirement for enzymic conversion of the reagent/component may contribute to the required specificity of response. As an example, the affinity of glucose-6-phosphatase for the added substrate glucose-6-phosphate is in the mM range.

Some substrates, for example glucose-6-phosphate, cannot normally cross the plasma membrane in mammalian cells. Therefore, methods making use of such substrates may work only if the cell membrane is permeabilised, preferably in cells fixed to a support, for example a slide.

By “target cell selective reagent” or “reagent capable of interacting selectively with one or more further components of the target cell” is meant a reagent that is selective for the target cell (in particular, a component thereof) in the context of performing the method. As noted above, it is preferred that the component interacts with at least 100-fold higher affinity (and preferably at least 500-fold, or at least 1000-fold, or at least 2000-fold higher affinity) with the relevant reagent than with other molecules that may be encountered by either the said component or reagent when performing the method. A reagent may be capable of interacting with cell types (for example non-recombinant cell types) other than the target cell (or positive control cell) but if such other cell types are substantially not present when performing the method, for example are not present in the test sample, the reagent may still be considered to be a target cell selective reagent. As noted above, a said other cell type may be used as a positive control cell, but this is not preferred. It is preferred that the reagent is not capable of interacting (or not capable of interacting to the same extent) with cell types other than the target cell type, for example under the conditions in which the method is performed. For example if the target cell is a bacterial cell (ie it is desired to identify/characterise or isolate any bacterial cell present in the test sample), it is preferred that the reagent is not capable of interacting (under the conditions in which the method is performed) with (non-recombinant) eukaryotic cells. If the target cell is an eukaryotic cell (ie it is desired to identify/characterise or isolate any eukaryotic cell present in the test sample), it is preferred that the reagent is not capable of interacting (under the conditions in which the method is performed) with (non-recombinant) prokaryotic cells. If the target cell is a malignant human cell, it is preferred that the reagent is not capable of interacting with (non-recombinant) non-malignant human cells, or with non-malignant human cells expected to be found in the test sample, for example in blood (for malignant cells other than leukaemic cells).

A component as discussed above is preferably a polypeptide or polynucleotide, but may alternatively be a lipid, carbohydrate, lipoprotein or glycoprotein. Similarly, a reagent may be a polypeptide or polynucleotide, or a lipid, carbohydrate, lipoprotein or glycoprotein; or a small molecule substrate. Desirable features of such a substrate are discussed, for example, in WO98/40746.

Conveniently, the said reagent is an antibody or fragment or derivative thereof.

Monoclonal antibodies which will bind to many cell-specific, for example cell surface, antigens (whether protein antigens or non-protein antigens) are already known but in any case, with today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Chimaeric antibodies are discussed by Neuberger et al (1988, 8th International Biotechnology Symposium Part 2, 792-799).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab₂ fragments may also be used as can genetically engineered antibodies and antibody fragments.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

It will be appreciated that when the target cell component (for example, in relation to fetal/embryonic cells, and adult liver component) is a receptor, the receptor, and hence the target cell (for example embryonic or fetal red blood cell) can be identified using a ligand for the receptor. For example, the cognate hormone is a ligand for a hormone receptor.

When the component is a polynucleotide, the reagent may also be a polynucleotide, for example a polynucleotide that is capable of hybridising selectively to the component.

By “selectively hybridising” is meant that the reagent nucleic acid has sufficient nucleotide sequence similarity with the said nucleic acid component that it can hybridise under moderately or highly stringent conditions, and preferably does not hybridise to other nucleic acids under the same conditions. As is well known in the art, the stringency of nucleic acid hybridization depends on factors such as length of nucleic acid over which hybridisation occurs, degree of identity of the hybridising sequences and on factors such as temperature, ionic strength and CG or AT content of the sequence. Thus, any nucleic acid which is capable of selectively hybridising as said is useful in the practice of the invention.

Nucleic acids which can selectively hybridise to the said nucleic acid component include nucleic acids which have >95% sequence identity, preferably those with >98%, more preferably those with >99% sequence identity, over at least a portion of the nucleic acid with the said nucleic acid component. As is well known, eukaryotic genes usually contain introns such that, for example, a mRNA or cDNA derived from a gene would not match perfectly along its entire length with the relevant eukaryotic genomic DNA but may nevertheless be useful as a reagent according to the present invention. Thus, the reagent may be a nucleic acid which selectively hybridises to a mRNA or cDNA but may not hybridise to the equivalent gene. For example, nucleic acids which span the intron-exon boundaries of the relevant gene may not be able to selectively hybridise to the equivalent mRNA or cDNA.

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is >500 bases is:

6×SSC (saline sodium citrate)

0.5% sodium dodecyl sulphate (SDS)

100 μg/ml denatured, fragmented salmon sperm DNA

The hybridisation is performed at 68° C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of sodium citrate in 800 ml of H₂O. Adjust the pH to 7.0 with a few drops of a 10 N solution of NaOH. Adjust the volume to 1 litre with H₂O. Dispense into aliquots. Sterilise by autoclaving.

An example of a typical hybridisation solution when a nucleic acid is immobilised on a nylon membrane and the probe is an oligonucleotide of between 15 and 50 bases is:

3.0 M trimethylammonium chloride (TMACl)

0.01 M sodium phosphate (pH 6.8)

1 mm EDTA (pH 7.6)

0.5% SDS

100 μg/ml denatured, fragmented salmon sperm DNA

0.1% non-fat dried milk

The optimal temperature for hybridization is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50° C.; for 19-mers, it is 55-57° C.; and for 20-mers, it is 58-66° C.

The reagent may be a nucleic acid which may be used to amplify DNA from the relevant genomic DNA, mRNA or cDNA (for example formed by reverse transcription of mRNA) by any of the well known amplification systems such as those described in more detail below, in particular the polymerase chain reaction (PCR).

It is preferred that PCR is used in the methods of the invention, for example PCR amplification of a single cell template sample.

Suitable conditions for PCR amplification include amplification in a suitable 1× amplification buffer:

10× amplification buffer is 500 mM KCl; 100 mM Tris.Cl (pH 8.3 at room temperature); 15 mM MgCl₂; 0.1% gelatin.

A suitable denaturing agent or procedure (such as heating to 95° C.) is used in order to separate the strands of double-stranded DNA.

Suitably, the annealing part of the amplification is between 37° C. and 65° C. The optimum temperature may be about 50° C. for many primer pairs but this may vary, as know to those skilled in the art.

A temperature of 72° C. may be used for the extension phase of the amplification when a thermostable polymerase is used, such as Taq polymerase.

Although the nucleic acid which is useful in the methods of the invention may be RNA or DNA, DNA is preferred. Although the nucleic acid which is useful in the methods of the invention may be double-stranded or single-stranded, single-stranded nucleic acid is preferred under some circumstances such as in nucleic acid amplification reactions.

The nucleic acid which is useful in the methods of the invention may be any suitable size. However, for certain diagnostic, probing or amplifying purposes, it is preferred if the nucleic acid has fewer than 10 000, more preferably fewer than 1000, more preferably still from 10 to 100, and in further preference from 15 to 30 base pairs (if the nucleic acid is double-stranded) or bases (if the nucleic acid is single stranded). Single-stranded DNA primers, suitable for use in a polymerase chain reaction, are particularly preferred.

Typically, in a method of identifying a target cell, the reagent (binding moiety) is detectably labelled or, at least, capable of detection. Such techniques are well known to those skilled in the art. For example, the binding moiety is labelled with a radioactive atom or a coloured molecule or a fluorescent molecule or a molecule which can be readily detected in any other way. The binding moiety may be directly labelled with a detectable label or it may be indirectly labelled. For example, the binding moiety may be an unlabelled antibody which can be detected by another antibody which is itself labelled. Alternatively, the second antibody may have bound to it biotin and binding of labelled streptavidin to the biotin is used to indirectly label the first antibody.

Typically, in a method of isolating target cells, the reagent (binding moiety) is immobilised on a solid support so that the target cells can be isolated by affinity binding. Conveniently, the solid support comprises any suitable matrix such as agarose, acrylamide, Sepharose (a trademark) and Sephadex (a trademark). The solid support may also be a solid substrate such as a microtitre plate or the like.

Advantageously, the binding moiety is magnetically labelled (either directly or indirectly) such that, when bound, the target cell can be separated from the rest of the sample upon provision of a suitable magnetic field. Microbeads used for magnetic cell sorting are often termed MACS colloidal super paramagnetic microbeads.

Target cells labelled in this way may be sorted by magnetic activated cell sorting (MACS).

Suitably, the binding moiety is labelled with a fluorescent molecule (either directly or indirectly) and the target cells are isolated using a fluorescence activated cell sorter (FACS). FACS methods are suitable for use with eukaryotic and prokaryotic cells; see, for example Rathman et al (2000) The development of a FACS-based strategy for the isolation of Shigella flexneri mutants that are deficient in intercellular spread. Mol Microbiol 35, 974-990.

Properties of Control Cells

A positive control cell is typically a non-pathogenic, non-malignant (non-harmful) cell, which is preferably recombinant, with the relevant isolation/identification/characterisation-conferring properties of the target cell (ie comprising a characterising component of the target cell which is the basis for the isolation/identification/characterisation method to be used). The positive control cell optionally comprises further components present in the target cell, conferring a genetic, and/or protein, lipid or carbohydrate constitution, of environmental, clinical, veterinary or other diagnostic interest in relation to the target cell. It is preferred that the positive control cell is recombinant in relation to the characterising component and/or said further component(s) shared with the target cell.

Positive control cells are preferably derived from a parent unicellular or multicellular lower organism cell, or from a cell or from a cell line derived from a multicellular higher organism. The parent cell is preferably not from the same species as the target cell, and still more preferably is not identical with the target cell. A positive control cell is preferably the result of modification of the parent cell so that the control cell comprises control components for isolation, identification and/or characterisation, for example for sub-typing, disease markers, and/or drug resistance markers. As discussed above, an identification and/or isolation marker is typically a component of the cell of interest that is preferably antigenic and preferably an outer surface or (less preferably) secreted component. It will be appreciated that the components of a target or control cell include secreted components of those cells. As discussed above, the marker component may preferably be a protein or part thereof but may alternatively (or in addition) be a carbohydrate or lipid moiety (including glycoproteins and lipoproteins). The marker component may also be a polynucleotide with a sequence characteristic of the target cell (or complementary to such a sequence), for example coding for a characteristic mutation and/or deletion, or the corresponding normal sequence, and/or chromosome of interest, and/or characteristic of the species or sub-type of interest. The control cell may further comprise the equivalent portions of rRNA or mRNA or polypeptide.

Positive control cells comprising/expressing polynucleotides/polypeptides (including lipoproteins and glycoproteins) of interest may be made using techniques well known to one skilled in the art. Suitable methods are described in Sambrook et al (1989) Molecular Cloning, A Laboratory Manual, Cold Spring Harbor Laboratory, Cold Spring Harbor, N.Y. Further exemplary techniques useful in constructing expression vectors and transforming appropriate host cells leading to the expression of the encoded polypeptide are disclosed, for example, in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

A negative control cell for use in conjunction with a particular positive control cell is typically identical to the positive control cell with the exception of the characterising component and optionally any said further component(s) shared by the positive control cell and target cell. The negative control cell is typically derived from the parent cell from which the positive control cell is derived and preferably is identical to the parent cell.

A positive or negative control cell is preferably non-virulent. The provision of non-virulent control cells is of particular benefit in relation to control cells for pathogenic microorganisms. The use of a non-virulent cell as an isolation/diagnostic control for a pathogenic organism obviates the need for stores of pathogenic and potentially virulent pathogens in routine diagnostic laboratories, and prevents the risk of contamination during transportation between laboratories.

Although not essential, it is preferred that the control cell is of substantially the same size and shape, and has similar biophysical properties (for example rigidity; presence of a cell wall or periplasmic membrane) as the target cell, in order to maximise the correspondence between the behaviour of the control and target cells during performance of the isolation, characterisation and/or identification method.

It is preferred that the characterising component (or further component(s)) has the same or similar subcellular distribution pattern in the control cell as in the target cell. This may be particularly useful if recognition of a spatial pattern (either by manual or automated pattern recognition methods) is important in distinguishing true positives from background “noise” for a particular test procedure.

Examples of suitable parent cells for the preparation of control cells include Escherichia coli (preferably non-pathogenic or disabled), Chinese hamster ovary (CHO) or Saccharomyces pombe (preferably non-pathogenic or disabled) cells. Further examples include H4IIe, Cos7, V79, 3T3 and the like (available from, for example, European Collection of Cell Cultures (ECACC), CAMR Centre for Applied Microbiology & Research, Porton Down, Salisbury, Wiltshire, UK, SP4 0JG), Lactobacillus sp, Bacillus subtilis, Pseudomonas sp, (all preferably non-pathogenic or disabled) and other preferably non-pathogenic bacteria, still more preferably those for which genomic sequence data is available.

Uses of Control Cells

The present invention relates to the concept of using model control cells as cellular controls in assays and in kits for performing such assays, and in allowing further diagnostic assays or approaches to be developed. It enables diagnosis to be carried out on individual cells by providing a way of validating the assay ie providing the necessary means of checking that the assay has been performed correctly. Without the control cells, it is not possible to determine whether the isolation and/or detection steps have been performed correctly; a negative result is therefore meaningless and quantitation is similarly impossible.

Uses of Control Cells in Relation to Lower Organism Diagnostics

Control cells may be used in accordance with the present invention in relation to isolating, identifying and/or characterising lower organism cells. By a “lower organism cell” is meant any cell from a species considered of lower evolutionary status than a mammal. The lower organism may be a pathogen of man, or of other mammals.

By a pathogen is meant an organism, which is detrimental to the health or wellbeing of the individual organism, preferably plant or mammal or fish, still more preferably human, or detrimental to others in the community. It may be able to survive and may be present in the environment, or in another (possible unaffected) species, or within an individual of the species affected.

It will be apparent that control cells may be used in cellular assays performed in a wide range of environments and for detecting a wide range of types of lower organism cells.

For example, a control cell may comprise a component characteristic of a pathogen of fish or crustaceans. Such control cells may be useful in monitoring or diagnostic tests on fish farms, for example salmon or cod fish farms, as will be evident to those skilled in the art. The pathogen may be harmful only to fish and/or crustaceans, or may also be pathogenic to other organisms, for example to man. Examples of fish pathogens include strains of the bacterium Aeromonas salmonicida, which cause a variety of diseases in fish, particularly farmed fish; rhabdoviruses, which cause infectious haemopoietic necrosis of salmonid fish, a major problem in farmed fish; haemorraghic septicaemia virus in cultivated turbot; Vibrio spp, which cause enteritis in Japanese flounder; herpes virus, which causes viral epidermal hyperplasia in Japanese flounder; agents contributing to enteric red mouth, cold water disease and furunculosis, which are frequent diseases of farmed trout. Examples of molecular characteristics of fish pathogens are given, for example, in Cascon et al (2000) A major secreted elastase is essential for pathogenicity of Aeromonas hydrophila. Infect & Immun 68, 3233-3241 (A hydrophila is the leading cause of fatal hemorrhagic septicemia in rainbow trout); Saulnier et al (2000) Rapid and sensitive PCR detection of Vibrio penaeicida, the putative etiological agent of Syndrome 93 in New Calcdonia. Des Aquat Org 40, 109-115; Wagner et al (1999) Monoclonal antibodies against AsaP1, a major exotoxin of the fish pathogen Aeromonas salmonicida subsp achromogenes, and their application in ELISA. J Appl Microbiol 87, 620-629.

The control cell may comprise a component characteristic of a plant pathogen or disease state. This may become more important as organic farming expands with a concomitant rise in plant disease and pathogens. Examples of plant pathogens include tobacco mosaic virus; maize streak virus; rice yellow mottle virus; banana brunchy top virus; tomato yellow leaf curl geminivirus; blossom blight, which affects apples and pears and is secondary to infection with the bacterium Erwinia amylovora; xylem-limited bacterial pathogens, for example Pierce's disease (grapes) secondary to the bacteriaum Xylella fastidiosa; Clavibacter michiganensis, for example spp Sepedonicus, which cause bacterial ring rot in potatoes.

Control cells may be used in environmental testing, for example testing of water supplies. The identification of small numbers of organisms in large volumes of fluid is desirable in such testing. Tests may be applied to water in, for example, reservoirs, rivers, pipes and conduits, tanks, sea water or effluent discharges. Examples of types of organisms which currently require testing in these large volumes of water include algae and cyanobacteria (toxic blooms) in reservoirs, shellfish and fish farms, Crytosporidium in water supplies, Legionnella in water supplies and unknown organisms in water supplies especially in hospitals and other public buildings.

Control cells may also be useful in tests for pathogens at various points in the food chain, for example monitoring public health measures in abattoirs, butchers, food processors and manufacturers, milk producers and the like. Suitable control cells may comprise a “universal” bacterial component, as indicated discussed below or a “universal” fungal component, or one or more components characteristic of a narrower class of organisms.

For example, if the assay is to be used to identify (which may include quantify) bacterial cells in general, a control cell comprising a (preferably recombinant) component present in all bacterial cells but not present in mammalian cells may be used as a control for an assay based on that component (characterising component). Such a component may comprise, for example, a polynucleotide having a conserved ribosomal RNA sequence found in all bacterial cells, ie a sequence described as a “universal” sequence in, for example, U.S. Pat. No. 5,994,066, incorporated herein by reference.

Thus, suitable control cells may be eukaryotic cells comprising “universal” bacterial components that are not present in eukaryotic cells, for example bacteria-specific cell surface components, and/or cell surface components which are present on selected bacteria of interest in a particular testing situation, for example as outlined above.

The control cell may comprise more than one, for example several, characterising components from different possible target cells, each reacting with a different target cell selective reagent, so that the cell can be used as a control for assays for more than one type of cell (ie may be a multi-identity control cell).

Control cells may be used in relation to the following exemplary organisms: Chlamydia; Mycoplasma; Ureaplasma; Meningococcus; Crytosporidium; Haemophilus influenzae; Staphylococcus sp; Streptococcus sp, including Pneumococcus; Escherichia coli; Heliobacter; Vibrio cholera; Klebsiella; Pseudomonas sp; cytomegalovirus; swine fever; gut parasites, for example hookworm.

Examples of fungal or yeast cells are Pichia, Saccharomyces, Kluyvermyces, Candida, Torulopsis, Hansenula, Schizosaccharomyces, Citeromyces, Pachysolen, Debaromyces, Metschunikowia, Rhodosporidium, Leucosporidium, Botryoascus, Sporidiobolus, Endomycopsis and the like. Control cells for such fungal or yeast cells may be useful. Further, cells of these types may be useful as host cells for preparing cells suitable for use as control cells in relation to other cell types, for example in relation to bacterial or higher eukaryotic cell types.

Chitin is a polysaccharide present in the cell wall of fungal cells (see, for example, J Food Protection (1996) 59, 73-81) and may be a suitable “universal” fungal antigen. Components of water-soluble extracellular secretions of fungal cells may be useful in detecting the presence of fungal cells and in providing information at the genus or species level. Heat stable polysaccharides tend to be specific for one or more genus of fungi. Examples of fungal specific monoclonal antibodies are described in Clinical Otolaryngology (1997) 22, 275-283, where they are used to detect fungi by immunofluorescent microscopy in ear swabs. Antisera against mycelia-soluble antigens which cross-react with antigens from other genera are described in Int J Food Microbiol (1997) 38, 191-200.

Examples of pathogenic yeast (for which it may be useful to have control cells) include pathogenic yeast of any one of the genera Candida spp, Blastomyces spp, for example B. dermatitidis, Coccidioides spp, for example C. immitis, Histoplasma spp, for example H. capsulatum, Sporothrix spp, for example S. schenckii, Aspergillus spp, for example A. fumigatus, A. flavus, A. niger, Phialophora compacta (Fonsecaea compacta), P. pedrosoi (F. pedrosi), P. verrucosa, Cladosporium carrionii, Rhinocladiella aquaspersa, Cryptococcus spp, for example C. neoformans, Cephalosporium spp, Fusarium spp, Histoplasma spp, for example H. capsulatum, Pneumocystis carinii, Rhizopus spp, Rhizomucor spp, Madurella spp, for example M. mycetomatis, M. grisea, Pseudallescheria boydii, Paracoccidioides spp, for example P. brasiliensis, Prototheca spp, for example P. wickerhamii, Epidermophyton spp, Microsporum spp, Trichophyton spp, and Malassezia spp, for example M. furfur (Pityrosporum orbiculare).

Examples of other pathogenic fungi include pathogenic fingi of the genera Aspergillus, including Aspergillus fumigatus, Cryptococcus, including Cryptococcus neoformans, and Histoplasma, including Histoplasma capsulatum. Control cells for such fungal or yeast cells may be useful.

The following references give examples of assays and molecular markers that may be useful as characterising or further components: de Souza et al (2000) Tagging of genes involved in multidrug resistance in Aspergillus nidulans 263, 702-711; Turin et al (2000) Fast, simple and highly sensitive double-rounded polymerase chain reaction assay to detect medically relevant fungi in dermatological specimens. Eur J Clin Invest 30, 511-518; Henry et al (2000) Identification of Aspergillus species using internal transcribed spacer regions 1 and 2 Clin Microbiol 38, 1510-1515; Zimmerman et al (2000) Ultra fast identification of Aspergillus species in pulmonary cytology specimens by in situ hybridization. Int J Mol Med 5, 427-429; Espinel-Ingroff et al (1999) Evaluation of DNA-based typing procedures for strain categorization of Candida spp. Diag Microbiol Infect Dis 33, 231-239; Reyes-Montes et al (1999) Relatedness analyses of Histoplasma capsulatum isolates from Mexican patients with AIDS-associated histoplasmosis by using histoplasmin electrophoretic profiles and randomly amplified polymorphic DNA patterns. J Clin Microbiol 37, 1404-1408; Jackson et al (1999) Species identification and strain differentiation of dermatophyte fungi by analysis of ribosomal-DNA intergenic spacer regions. J Clin Microbiol 37, 931-936; Hong et al (1998) Optimisation of Candida albicans typing by pulsed-field gel electrophoresis. Br J Biomed Sci 55, 231-237; Cresti et al (1999) Molecular typing of Candida spp. by random amplification of polymorphic DNA and analysis of restriction fragment length polymorphism of ribosomal DNA repeats. Microbiologica 22, 41-52; Kolaczkowski et al (1998) In vivo characterization of the drug resistance profile of the major ABC transporters and other components of the yeast pleiotropic drug resistance network. Microbiol Drug Resist Mech Epid Dis 4, 143-158; Poonwan et al (1998) Genetic analysis of Histoplasma capsulatum strains isolated from clinical specimens in Thailand by a PCR-based random amplified polymorphic DNA method. J Clin Microbiol 36 3073-3076; Colonna-Romano et al (1998) Identification and isolation by DDRT-PCR of genes differentially expressed by Histoplasma capsulatum during macrophages infection. Microbiol Path 25, 55-66; Kappe et al (1998) Molecular probes for the detection of pathogenic fungi in the presence of human tissue. J Med Microbiol 47, 811-820; Vanittanakom et al (1998) Specific identification of Penicillium marneffei by a polymerase chain reaction hybridization technique. Med Mycol 36, 169-175; Mannarelli & Kurtzman (1998) Rapid Identification of Candida albicans and other human pathogenic yeasts by using short oligonucleotides in a PCR system. J Clin Microbiol 36, 1634-1641; Montone & Litzky (1995) Rapid method for detection of Aspergillus 5S ribosomal-RNA using a genus-specific oligonucleotide probe. Am J Clin Pathol 103, 48-51; Xia et al (1992) Development of monoclonal antibodies specific for Pyricularia grisea, the rice blast pathogen. Mycol Res 96, 867-873.

When testing for fungal cells, it may be desirable for the control cell to be a recombinant bacterial cell, and vice versa.

A control cell may be provided using techniques of molecular biology well known to those skilled in the art.

For example, the following outline protocol may be used in preparing a control cell for a particular prokaryotic or lower eukaryotic cell, for example a pathogenic cell type. Isolate genetic material from the cell type of interest; clone into appropriate expression vectors; express in appropriate non-pathogenic cell type (preferably an organism of a different species to the pathogenic organism of interest), for example a disabled E. coli strain; establish new model strain; make appropriate antibodies (by using the expressed material (which may be expressed with a tag (subsequently removed/masked) such as GST, His, FLAG, myc to aid purification) as an antigen; select appropriate antibodies (for example by logical prediction/selection and/or by systematic testing, for example testing for non-reactivity against non-target cells); develop identification, isolation and/or characterisation methods (using the control cell as a positive control and preferably also a negative control cell) using the antibody; test efficacy of antibodies on target cells.

It is desirable to use immunohistochemical or other in situ technologies to visualise the components in the positive and negative control cells using the antibodies, in order to assess the antibodies. This method has the advantages of showing whether the antibodies bind, and where the reacting antigens are located in the positive control cell It allows non-reactivity to the negative control cell to be checked. Where the molecule of interest appears to be on the cell surface, the test can be repeated in both intact and permeablised cells, in order to determine whether the antigenic epitopes of interest are on the outside of the cell. Cells/antigens in which the epitopes are on the outside may be preferred in relation to isolation methods, although others may also be effective using FACS and similar technology. The utility of the antibodies on cell isolation procedures may then be tested.

Analogous techniques in which nucleic acid hybridisation is checked may be used when the characterising or further component is a nucleic acid.

When quantitation is desired, a number of positive but different control cell lines may be required, each expressing different levels of the material of interest. To achieve this, different expression vectors may be required, and a large number of potential clones may need to be screened to allow selection of a desired range of expression levels in the control cells.

To obtain cell surface expression of a molecule or portion thereof in a different cell type, for example a bacterial protein in a mammalian cell, it may be important to use a vector which contains an in-frame targeting sequence, which normally targets components, for example polypeptides, to the desired portion (for example the cell surface) of the control cell.

It may also be necessary to adjust the codon usage when expressing a polypeptide in a different cell type. As well known to those skilled in the art, codon usage differs between organisms and sometimes (but not always) this causes problems in expressing a polypeptide in cells of other species. If this occurs it may be necessary to change some or all of the codons in the polynucleotide in an expression vector to those used in the new host.

The process is repeated until the control cell expresses all the necessary components for isolation (preferably outer surface or secretory components); for identification (preferably proteins or other antigenic material); and for determining the strains and their individual antibiotic resistance profile, and/or virulence factors, and/or therapeutic sensitivity profile (preferably DNA, RNA, protein or carbohydrate of interest).

The control cell may comprise one or more components derivable from the target cell type, for example as indicated above.

Suitable methods for identifying polynucleotides encoding for immunogenic or virulence-determining molecules are described, for example, in WO96/17951, incorporated herein by reference, which describes methods for identification of genes involved in the adaptation of a microorganism to its environment, particularly the identification of genes responsible for the virulence of a pathogenic microorganism. WO99/01473 (incorporated herein by reference) also relates to such methods applied to Staphylococcus aureus and identifies S. aureus virulence genes.

U.S. Pat. No. 5,994,066 (incorporated herein by reference) describes methods by which species-specific, genus-specific, “universal” and antibiotic resistance gene-specific DNA probes and amplification primers for bacteria may be identified. The particular probes and amplification primers described may be useful in the practice of the present invention. For example, genes and encoded proteins indicated in Table 8 of U.S. Pat. No. 5,994,066 may be useful as universal, genus or species-specific detection or isolation probes using a method of the invention. In addition, the methods described may be useful in identifying further probes (polynucleotide or, for example, polypeptide) and amplification primers.

U.S. Pat. No. 6,001,565 (incorporated herein by reference) also relates to species specific and “universal” DNA probes and amplification primers useful in identifying bacterial pathogens and antibiotic resistance genes. Portions of such genes and nucleotide sequences may be incorporated into a control cell for use in methods of the present invention.

In particular, a control cell for cell isolation, characterisation and/or identification in relation to Haemophilus influenzae may express all or a portion of the outer membrane protein P1 (encoded by the gene ompP1; SEQ ID No 26 of U.S. Pat. No. 5,994,066) and/or the outer membrane protein P6 (encoded by the gene ompP6; SEQ ID No 178 of U.S. Pat. No. 5,994,066).

A control cell for cell isolation, characterisation and/or identification for Proteus mirabilis may express all or a portion of a lipoprotein encoded by the gene lpp (SEQ ID No 181 of U.S. Pat. No. 5,994,066).

A control cell for cell isolation, characterisation and/or identification for Klebsiella pneumoniae may express all or a portion of the citrate carrier protein encoded by the gene cit (SEQ ID No 182 of U.S. Pat. No. 5,994,066) or of the outer membrane protein II (encoded by the gene ompA (SEQ ID No 11 of U.S. Pat. No. 5,994,066).

A control cell for cell isolation, characterisation and/or identification for Pseudomonas aeruginosa may express all or a portion of the outer membrane protein H1 (encoded by the gene oprH; SEQ ID No 19 of U.S. Pat. No. 5,994,066) or Exotoxin A.

A control cell for cell isolation, characterisation and/or identification of any bacterial cell may express all or a portion of the H⁺-transporting ATPase β-subunit (encoded by gene atpD; SEQ ID No 187 of U.S. Pat. No. 5,994,066).

Table 9 of U.S. Pat. No. 5,994,066 lists selected antibiotic resistance genes. Portions of such genes may be incorporated into a control cell for use in methods of the present invention.

U.S. Pat. No. 5,919,640 (incorporated herein by reference) describes an adhesin produced by the pathogen Streptococcus suis, which causes serious disease in pigs and may also be infectious to man. Vaccination and diagnostic methods making use of the adhesin are described. A control cell for use in methods of cell isolation, characterisation and/or identification for S. suis may comprise all or a portion (for example the unique N-terminal part of the polypeptide or a fragment thereof) of the adhesin or a polynucleotide encoding all or a portion of it. U.S. Pat. No. 5,955,078 (incorporated herein by reference) relates to matrix binding proteins from Staphylococcus aureus or Staphylococcus epidermidis, particularly fibronectin binding proteins (FbpA, FbpB and the polypeptide shown in SEQ ID No 10 of U.S. Pat. No. 5,955,078), and antibodies thereto. A control cell for use in methods of cell isolation, characterisation and/or identification of Staphylococci, for example S. aureus or S. epidermidis, may comprise all or a portion of such a matrix binding protein or a polynucleotide encoding all or a portion of it. Suitable portions are described in U.S. Pat. No. 5,955,078 and may include the fibronectin binding domain, a region of about 38 amino acids that is repeated three time (D1-D3 region) and partially repeated a fourth time (D4 region). A suitable polypeptide may be shown in SEQ ID No 11, No 6, 7 and/or 8 of U.S. Pat. No. 5,955,078.

WO98/02547 (incorporated herein by reference) describes polynucleotide sequences found in Neisseria meningitidis but not in Neisseria gonorrhoeae or in Neisseria lactamica. Control cells comprising one or more such polynucleotide sequence and/or polypeptide encoded by such polynucleotide sequence may be useful in methods of cell isolation, characterisation and/or identification for N. meningitidis bacteria, and may therefore be useful in methods of diagnosing meningococcus induced infections and meningitis.

U.S. Pat. No. 6,013,514 (incorporated herein by reference) relates to the D15 outer membrane protein of Haemophilus influenzae. Control cells comprising such a polypeptide or a portion thereof (and/or a polynucleotide sequence encoding the polypeptide or a portion thereof) may be useful in methods of cell isolation, characterisation and/or identification for H. influenzae, for example in the diagnosis of bacterial meningitis, for example using methods based on those described in U.S. Pat. No. 6,013,514.

U.S. Pat. No. 5,994,090 (incorporated herein by reference) describes a method of detecting mycoplasma in which an antibody specific for GGPL-III, a phosphocholine-containing glycoglycerolipid specific to Mycoplasma ferimentans, is used. A control cell for use in relation to such a method may comprise such a phosphocholine-containing glycoglycerolipid. Such a cell may be obtained by testing cells recombinant for M. fermentans genetic material for expression of the phosphocholine-containing glycoglycerolipid as described in U.S. Pat. No. 5,994,090.

U.S. Pat. No. 5,827,651 (incorporated herein by reference) relates to nucleic acid probes and methods useful in detecting non-viral organisms, particularly mycobacteria, various bacteria and fungi. The probes relate to rRNA sequences characteristic of particular groups of organisms. Portions of such nucleotide sequences may be incorporated into a control cell for use in a method of the present invention. Such a method may be based on a method described in U.S. Pat. No. 5,827,651.

U.S. Pat. No. 5,788,962 (incorporated herein by reference) describes Mycoplasma hyopneumoniae (which causes enzootic pneumonia in swine) P65 surface antigens and diagnostic tests making use of such antigens. Other surface lipoproteins are also mentioned. A control cell useful in methods of isolation, characterisation and/or identification for M. hyopneumoniae may comprise such a surface antigen, for example the P65 polypeptide or a portion thereof. A control cell comprising a P65 epitope may be particularly useful in distinguishing M. lyopneumoniae from M. flocculare. For example, such a control cell may be useful in a diagnostic method based on those described in U.S. Pat. No. 5,788,962.

Examples of recombinant cells suitable for use as control cells in relation to bacterial, fungal, mycobacterial or plant or fish pathogen cell testing are indicated in the following table. It will be appreciated that the other combinations of control cell background, characterising and further components are also suitable as control cells. Components indicated as characterising components may also be used as confirmatory components (or vice versa), such that a control cell may have two or more components indicated in the table as characterising or confirmatory components. Interaction of a target cell selective reagent with the characterising component may be used in identifying, characterising and/or isolating the target or positive control cell. Clinical interest may be in relation to human and/or animals.

| | Charac- | Further | Further | |
|---|---|---|---|---|
| | terising | (confirmatory) | (diagnostic) | Control cell |
| Target cell | component | component | component | background |
| Bacterial cell | H⁺- | Translation | Bla gene | Saccharomyces |
| | transporting | elongation | sequence | pombe |
| | ATPase | factor EF-Tu | (β-lactam | |
| | β-subunit | (see U.S. Pat. No. | resistance) | |
| | | 5,994,066) | (see U.S. Pat. No. | |
| | | | 5,994,066) | |
| Haemophilus | Outer | D15 outer | A portion or | E. coli |
| influenzae | membrane | membrane | product of an | |
| | protein P1 | protein | antibiotic | |
| | | | resistance | |
| | | | gene/gene of | |
| | | | specific clinical | |
| | | | interest, eg | |
| | | | Ampicillin | |
| | | | resistance | |
| Klebsiella | Outer | | A portion of | Bacillus subtilis |
| pneumoniae | membrane | | product of an | |
| | protein P2 | | antibiotic | |
| | | | resistance | |
| | | | gene/genes of | |
| | | | specific clinical | |
| | | | interest, eg | |
| | | | Gentamicin | |
| | | | resistance | |
| H. influenzae | D15 outer | Outer | A portion or | E. coli |
| | membrane | membrane | product of an | |
| | protein | protein P1 | antibiotic | |
| | | | resistance | |
| | | | gene/gene of | |
| | | | specific clinical | |
| | | | interest eg | |
| | | | Choramphenicol | |
| | | | resistance | |
| Streptococcussuis | H⁺- | Fragment of an | A portion or | E. coli |
| | transporting | adhesin as | product of an | |
| | ATPase | described in | antibiotic | |
| | beta | U.S. Pat. No. | resistance | |
| | subunit | 5,919,640 | gene/gene of | |
| | | | specific clinical | |
| | | | interest eg | |
| | | | Penicillin | |
| | | | resistance | |
| Pseudomonas | Outer | | A portion or | B. subtilis |
| aeruginosa | membrane | | product of an | |
| | protein H1 | | antibiotic | |
| | | | resistance | |
| | | | gene/gene of | |
| | | | specific clinical | |
| | | | interest eg | |
| | | | Gentamicin | |
| | | | resistance | |
| Pyricularia | Conidia | Germ-tube | A portion or | S. pombe |
| grisea | surface | antigens eg 3E4 | product of an | |
| | antigens eg | | antibiotic | |
| | Mab4G11- | | resistance | |
| | reactive | | gene/gene of | |
| | antigen | | specific clinical | |
| | | | interest eg | |
| | | | rocagloal, | |
| | | | benzofurans, | |
| | | | Benlate, blasticin S | |
| | | | resistance | |

A further aspect of the invention provides a recombinant cell comprising combinations of the components indicated in the table.

The preferred combinations of markers in a control cell depends on the particular use intended for the control cell. For example, control cells may be required for the following purposes, for example in relation to lower organisms:

1) controls for identification of cell/organism types (generic controls);

2) controls for efficacy of antibodies (which are preferably exterior surface component-specific antibodies in isolation procedures), for example for use in a test isolation procedure using positive control cells expressing markers of choice, mixed with identical cells without the markers;

3) quality controls for the process of making and optimising diagnostic tests by companies;

4) quality controls for end users of such diagnostic test (ie controls for the laboratory technicians and/or automated machinery performing the test, to confirm that the correct cells have been identified, isolated and/or characterised);

5) quality controls for the assurance of the health care professionals making use of results of the diagnostic tests;

6) controls relating to the particular interest of the specific end-user, for example controls for resistance to particular antibiotics, or other information such as sub-type of organism, that the clinician may need.

Control cells may be required for the following purposes in relation to higher eukaryotic cells:

1) controls for identification of cell types. Positive control cells may be used as positive controls for a diagnostic process that uses microscopy to identify individual cells of interest, preferably using a slide based technology. Subsequent to the identification of the cell of interest, the determination of the relevant characteristics of the cell, preferably therapeutically relevant characteristics, and/or diagnostic test(s) can be slide or tube based.

2) controls for efficacy of antibodies (which are preferably exterior surface component-specific antibodies in isolation procedures) or other aspects of an isolation technique, for example for use in a test isolation procedure using small numbers of positive control cells expressing markers of choice, mixed with larger numbers of identical cells without the markers. In the routine clinical diagnostic laboratory, isolation of very small numbers of fetal or metastatic cells from bodily fluids containing relatively large numbers of other cell types is required.

3) quality controls for the process of making and optimising diagnostic tests by companies. For the manufacturer to design, optimise and quality control isolation and/or diagnostic kits where very small numbers of fetal or metastatic cells are being recovered from bodily fluids containing relatively large numbers of other cell types requires a readily available standard control, such as a few of the relevant positive control cells in a fluid containing large numbers of the negative control cell (for example parent cell used in preparing the positive control cell).

4) Quality controls for end users of such diagnostic tests. Controls for the laboratory technicians and/or automated machinery performing cell isolation steps are relevant positive control cells, preferably in the presence of the relevant negative control cell, for example immobilised on a slide. The positive control cells preferably contain a further marker of the cell type of interest that is different from the marker used for isolation. Preferably the negative control cells do not contain either the identification or isolation marker. Positive control cells with different levels of expression of the identification or isolation type characteristic allow detection limits to be determined.

5) Quality controls for the health care professionals involved in making the actual diagnosis. In the diagnosis of a specific disease using one or more single cell, a positive control cell containing the mutation of interest and/or the normal relevant species-specific control sequence, is used as a quality control for the particular diagnostic technique.

6) controls relevant to the diagnostic purpose of the particular end user, for example controls for qualities which are potentially informative for therapeutic management decisions, for example drug resistance of metastatic cells, oncogene expression and/or mutation patterns, or cell sub-types. Positive control cells would express the appropriate markers, for example cell sub-type specific markers, drug resistance markers and/or oncogene markers.

Uses of Control Cells in Relation to Higher Eukaryotic Cells

Control cells may also be used in relation to tests to isolate, identify and/or characterise cells from higher eukaryotic organisms. This includes higher eukaryotic organism cells that have been infected by a lower organism, for example a virus or a mycobacterium. Thus, the comments below may also be relevant to tests for detecting host cells infected with an organism mentioned above, particularly a virus.

By a “mammalian cell” is meant any cell from a mammal. When the target cell is a mammalian cell, it is preferably from a human, or a domesticated mammal, for example a mammal of commercial or agricultural importance or domestic animal. Suitably, the mammal is a horse, cow, sheep, pig, goat, dog, cat or the like.

Chinese hamster ovary (CHO) cells, for example, may be used in preparing controls for human cells. Their low adhesion in cell culture may make them particularly suitable for use as control cells in the methods of the invention.

Uses of Control Cells in Relation to Fetal Diagnostics

The present invention provides the use of control cells with the relevant properties of normal human embryonic/fetal red blood cells, optionally with further relevant properties of human embryonic/fetal red blood cells with an abnormal karotype and/or genetic constitution. Preferred model cells in this context are mammalian, non-human (for example rodent) cells comprising at least one (preferably two or more) human adult liver components (as discussed further below) and optionally a polynucleotide sequence associated with (or associated with the absence of) a human disease, condition, or trait, for example a polynucleotide sequence associated with cystic fibrosis.

Control cells may thus be used in assays for genetic disease in fetuses, particularly in relation to assays in which cells are isolated and/or analysed without culturing the cells. Such assays may not be feasible unless control cells are used, in accordance with the present invention. The cells for analysis may be recovered, for example, from amniocentesis and chorionic villus biopsies or may be recovered from the maternal bloodstream, as described below.

Control (model) cells may also be useful in refining identification, isolation, and/or diagnostic techniques involving embryonic/fetal red blood cells.

By an embryo is meant a conceptus of a developmental stage equivalent to up to and including 56 post-ovulatory days for a human conceptus. The embryo is preferably a human conceptus, still more preferably a human conceptus of up to and including 56 post-ovulatory days.

It will be appreciated that the basic pattern of haematological development, for example, in the embryo and fetus is the same for all mammals.

By a fetus is meant a conceptus of a developmental stage equivalent to greater than 8 weeks gestational age for a human conceptus. It is preferred that the fetus is a human conceptus, still more preferably a human conceptus of greater than 8 weeks gestational age. Methods of estimating gestational age are summarised in WO98/40746, incorporated herein by reference.

By a fetal/embryonic blood cell component is meant a component of a fetal/embryonic blood cell, preferably a red blood cell, still more preferably a nucleated red blood cell, which is substantially not otherwise present in other cells in the fluid, preferably adult blood, amniotic fluid or urine from a patient with haematuria, from which it is intended to isolate or identify the fetal/embryonic blood cell.

By a fetal/embryonic trophoblastic cell component is meant a component of a fetal/embryonic trophoblastic cell, which is substantially not otherwise present in other cells in the fluid, preferably adult blood, amniotic fluid or urine from a patient with haematuria, from which it is intended to isolate or identify the fetal/embryonic trophoblastic cell.

By a fetal/embryonic stem cell component is meant a component of a fetal/embryonic stem cell, which is substantially not otherwise present in other cells in the fluid, preferably adult blood, amniotic fluid or urine from a patient with haematuria, from which it is intended to isolate or identify the fetal/embryonic stem cell component.

A still further aspect of the invention provides a recombinant mammalian cell comprising two or more human adult liver components and optionally a polynucleotide sequence associated with, or associated with the absence of, a human disease, condition, or trait, for example a polynucleotide sequence associated with cystic fibrosis.

Conveniently, the two or more human adult liver components are expressed in the mammalian cell by recombinant means. Preferably, the polynucleotide sequence is present due to recombinant means.

WO98/40746 (incorporated herein by reference) describes methods of isolating or identifying embryonic or fetal red blood cells in a sample containing maternal blood cells and embryonic or fetal red blood cells or both, the method comprising determining which cell or cells contain or express an adult liver component, or isolating the cells which contain or express an adult liver component. Suitable adult liver components are defined and described, and include, for example, glucose-6-phosphatase. A control cell for use in a method according to WO98/40746 comprises an appropriate adult liver component, ie the same component on the basis of which the embryonic or fetal red blood cells are identified or isolated.

The methods of WO98/40746 are advantageous, for example because they allow fetal diagnosis to be performed using maternal blood samples. This offers less risk to the mother or fetus than techniques such as those involving amniocentesis.

Inclusion of cellular controls according to the present invention will aid interpretation of the results of methods such as those described in WO98/40746 and may allow improvements in the speed, sensitivity and reliability of such methods.

Thus, when the target cell is a fetal or embryonic red blood cell, it is preferred that the said characterising component is an adult liver component. Suitable components are described in WO98/40746. It is particularly preferred that the characterising component is a plasma membrane component, for example GLUT2, a P-glycoprotein, a MDRP (multidrug resistance protein), a MRP (multidrug resistance-like protein), γ-glutamyl transpeptidase, a lipoprotein receptor, an alkaline phosphatase (a hepatic plasma membrane protein), a bile salt transporter, a bile acid transporter, a hormone receptor, a MOAT (multiple organic ion transporter; equivalent to MRP), a bilirubin transporter or a bilirubin conjugate (eg bilirubin glucuronide) transporter (equivalent to MRP).

A suitable further component is glucose-6-phosphatase or UDP glucuronosyltransferase, as described in WO98/40746. Another suitable further component may be a component linked with a genetic disease or condition (including sex) or fetal abnormality. Examples of such other components are given in WO98/40746. The genetic disease may be a cancer, for example retinoblastoma.

It will be appreciated that the reason for analysing fetal or embryonic cells is frequently to investigate the genetic makeup of the fetus/embryo. Accordingly, inclusion of one or more component in a control cell which can act as a positive or negative control for particular genetic markers may be particularly desirable in relation to control cells for fetal/embryonic cells.

Conveniently, the component linked with a genetic disease or condition (including sex) or fetal abnormality will be a polynucleotide, as discussed in WO98/40746.

Examples of recombinant cells suitable for use as control cells in relation to fetal/embryonic cell testing are indicated in the following table. It will be appreciated that the other combinations of control cell background, characterising and further components are also suitable as control cells. Components indicated as characterising components may also be used as confirmatory components (and vice versa), such that a control cell may have two or more components indicated in the table as characterising components. The characterising component may be made use of in relation to isolation, characterisation and/or identification techniques.

| | | Further | Further | |
|---|---|---|---|---|
| | Characterising | (confirmatory) | (diagnostic) | Control cell |
| Target cell | component | component | component | background |
| Fetal/embryonic | GLUT2 | UDPGT1A1 | Mutation | V79 |
| cell in | | | linked with | |
| maternal | | | hypercholesterolaemia | |
| circulation | | | | |
| Fetal/embryonic | GLUT2 | Glucose-6- | Mutation | H4IIE |
| cell in | | phosphatase | linked with | |
| maternal | | | Huntingdon's | |
| circulation | | | Chorea | |
| Fetal/embryonic | MRP | Endoplasmic | Portion of a | Saccharomyces |
| cell in | | reticulum | chromosome of | |
| maternal | | transport | diagnostic | |
| circulation | | protein | interest, eg | |
| | | | human | |
| | | | chromosomes | |
| | | | 13, 17, 21 | |
| Fetal/embryonic | Alkaline | NADPH | Mutation or | Cos 7 |
| cell in | phosphatase | cytochrome | deletion of | |
| maternal | | P450 oxido- | ornithine | |
| circulation | | reductase | transcarbamylase | |
| | | | gene | |
| Trophoblastic | Murine IgG2a | Y chromosome | Mutation of | CHO |
| cell in | monoclonal | | Niemann Pick | |
| maternal | antibody H315 | | Disease, eg | |
| fluids | antigen | | R496L or | |
| | | | L302P | |
| Fetal/embryonic | | Y chromosome | Mutations of | 3T3 |
| cell in | | | Haemophilia | |
| amniotic fluid | | | A, eg G1111R | |
| | | | or E1885K | |
| Fetal/embryonic | CD34 (for | CD 38 negative | Mutations of | Saccharomyces |
| cell in | haemopoietic | | alpha- | |
| maternal | multipotent | | galactosidase A | |
| fluids | progenitor cells) | | causing | |
| | | | Fabry's | |
| | | | Disease, eg | |
| | | | Q327K or | |
| | | | W44X | |
| Fetal/embryonic | Transferrin | HbFetal gamma | Y chromosome | Cos 1 |
| cell in | receptor | chain | | |
| maternal | | | | |
| circulation | | | | |
| Fetal cell in | MDR | UDPGT1A1 | Mutation | CHO |
| maternal | | | linked with | |
| circulation | | | cystic fibrosiseg | |
| | | | deletion | |
| | | | F508 or R553X | |

By inclusion of a deletion we mean that the control cell comprises a portion of polynucleotide containing the version of the gene of interest with the deletion, which can be detected by techniques known to those skilled in the art, for example sizing or sequencing of PCR products, or using in situ probes. Preferably a second control cell comprising the equivalent portion of polynucleotide from the normal version of the gene (ie without the deletion) is also used.

In the case of isolation of fetal/embryonic stem cells from maternal fluid, it is preferable to select CD34 positive and CD38 negative cells. It is desirable that negative control cells express CD38 whilst positive control cells do not. The CD38 and CD34-specific reagents could each be either a target cell selective reagent or a further confirmatory reagent.

For fetal cell isolation controls, the most preferable control cell type would be mammalian, preferably not human, in which a human fetal or embryonic red blood cell plasma membrane component, preferably a protein or part thereof was expressed as an isolation control, with an independent confirmatory identification marker expressing another fetal/embryonic red blood cell component as an identification control. A disorder control eg for Trisomy 21 would require human chromosome 21 or a portion thereof. A disorder control in relation to cystic fibrosis would require the presence of nucleic acid, preferably DNA and optionally also RNA, containing the mutated sequence, insert or deletion associated with cystic fibrosis.

The present invention may facilitate earlier and safer diagnosis of fetal disorders, including potentially all chromosomal and single gene disorders.

It will be appreciated that control cells may beneficially be used with other methods of analysing fetal/embryonic cells in addition to those that make use of the expression of adult liver components. Examples of other methods are reviewed in WO98/40746.

Situations in which it is desirable to use positive control cells include the following: analysis of embryonic and fetal cells obtained from maternal blood, at fetal biopsy, at amniocentesis, at chorionic villus biopsy, or from other sources of amniotic fluid; analysis of cells taken from dividing embryos in relation to in vitro fertilisation.

Other Uses in Relation to Mammalian Cells

Control cells may be useful in relation to analysis of cells in body fluids in normal or diseased states (including tumour cells) preferably in blood, cerebrospinal fluid, lymphatic fluid, synovial fluid, tears, pleural, peritoneal, pericardial, gastrointestinal tract, biliary system, urinary tract (preferably urine), semen, cervical and vaginal fluids. Control cells may also be useful in analysis of cells in samples from the urogenital, respiratory, gastrointestinal tracts by aspirates, smears and brushings, or from solid tissue samples. It will be appreciated that such samples may also be suitable and useful in relation to investigating infection by lower organisms, for example bacteria and fungi.

WO98/53102 (incorporated herein by reference) relates to methods for differentiating swine that are genetically susceptible to diseases associated with F18 E. coli infection from resistant swine. Infection leads to oedema disease and postweaning diarrhea. Nucleotide polymorphisms associated with susceptibility are described. Portions of such nucleotide sequences may be incorporated into a control cell for use in methods of the present invention, applied, for example, to methods described in WO98/53102.

Control cells may be used in relation to tests for infected cells. Such cells may have surface or internal components that are not present in uninfected cells. Tests for infected cells are particularly important in relation to organisms that are found or replicate exclusively or predominantly in a host cell. Thus, these tests are particularly important in relation to viral infections, mycobacterial infections and intracellular parasites such as Plasmodium (which causes malaria), as will be well known to those skilled in the art.

Examples of viral infections include those mentioned above in relation to fish and plant pathogens; Hepatitis A,B,C,D,E; Herpes simplex; cytomegalovirus; the causative agent of swine fever; Human Immunodeficiency virus (HIV); rubella and polio. Suitable components, for example antigens expressed on the surface of an infected cell, will be apparent to the skilled person in view of the teaching given herein.

WO98/36089 (incorporated herein by reference) relates to test kits for diagnosing tuberculosis. It describes a 40 kDa antigen that is present in vivo as a hexamer and which does not cross-react with the vaccine strain M. bovis BCG. Thus, this antigen may be particularly suitable for distinguishing an infected individual from a BCG-vaccinated individual. A control cell suitable for use in a method of detecting mycobacterial presence or infection may comprise the said 40 kDa antigen or a portion thereof, or a polynucleotide encoding same. Such a control cell may be useful as a further component of the test kits or methods of WO98/36089. Control cells comprising other antigens described in WO98/36089 may also be useful in methods of isolating and/or identifying mycobacterial cells.

WO98/53076 (incorporated herein by reference) describes compounds and methods for diagnosing tuberculosis. Polypeptides that contain at least one antigenic portion of one or more Mycobacterium tuberculosis proteins, and DNA sequences encoding such polypeptides are described. Methods of identifying suitable antigens and nucleic acids encoding them are described. Control cells useful in isolating and/or detecting M. tuberculosis may comprise one or more antigenic molecule or nucleic acid encoding such a molecule as described in WO98/53076.

U.S. Pat. No. 5,770,719 (incorporated herein by reference) also relates to mycobacterial immunogens, particularly membrane-associated immunogens. The sequence of a 79 kDa membrane polypeptide, identified as a membrane bound, ion-motive ATPase from M. bovis BCG is given. Methods of identifying genes encoding immunogenic polypeptides are also described. Portions of such genes and nucleotide sequences may be incorporated into a control cell for use in methods of the present invention, particularly in relation to methods of diagnosing tuberculosis.

WO99/24577 (incorporated herein by reference) relates to nucleic acid fragments and polypeptide fragments derived from M. tuberculosis, including fusions of such polypeptides. Control cells comprising one or more of such fragments may be useful in assays for detecting or characterising M. tuberculosis. In particular, SEQ ID Nos 175, 179, 181 and 185 relate to antigenic polypeptides which may be desirable to include in a control cell. Examples of further suitable antigens are listed in the table on page 4 of WO99/24577. Methods useful in identifying useful antigens and polynucleotides encoding them are also described. Polypeptides which are lipidated, as described, for example, on page 16, may be particularly suitable for inclusion in a control cell.

WO98/04711 and U.S. Pat. No. 6,010,855 (both incorporated herein by reference) describes an exported protein of M. tuberculosis termed DES. It also mentions other exported proteins (with references), for example a 19 kDa M. tuberculosis lipoprotein, the ERP protein similar to the M. leprae 28 kDa antigen and a stearoyl-Coenzyme A desaturase enzyme system (which may include the DES protein). The DES protein is encoded by the des gene and appears to be highly antigenic and is conserved amongst mycobacterial species: the M. tuberculosis DES protein reacts with human sera from tuberculosis and leprosy patients but not with sera from tuberculous cattle. A control cell comprising the DES protein or a portion thereof may be useful in isolating and/or detecting mycobacterial cells, particularly in humans, and may therefore be useful in diagnostic tests for tuberculosis or leprosy.

U.S. Pat. No. 5,916,558 (incorporated herein by reference) relates to polypeptides and polynucleotides useful in the diagnosis of tuberculosis. Control cells useful in assays for isolating and/or detecting mycobacteria, for example for diagnosing tuberculosis may comprise one or more such polypeptide or polynucleotide. Polypeptides which elicit a humoral response in lepromatous leprosy patients (30 kDa and 31 kDa antigens) and tuberculoid leprosy patients (32 kDa antigen) are also described. Thus control cells comprising such polypeptides (or fragments thereof) or polynucleotides encoding them may be useful in diagnostic tests for leprosy. Control cells comprising one or more nucleotide sequences shown in Table 1 of U.S. Pat. No. 5,916,558 may be useful in methods of distinguishing M. tuberculosis from other bacterial strains, in particular from mycobacterial species M. vmarinum, M. scrofulaceum, M. gordonae, M. szulgai, M. intracellulare, M. xenopi, M. gastri, M. nonchromogenicum, M. terrae, M. triviale, M. bovis, M. kansasii, M. avium, M. phlei and M. fortuitum.

WO98/31387 (incorporated herein by reference) identifies two mycobacterial antigens which may be linked (or the presence of an immune response to the antigen may be linked) with the activity of an infection. Such antigens may be useful in distinguishing patients with active mycobacterial disease, for example active tuberculosis, from patients who have been exposed to mycobacteria but who have not developed active disease. Other mycobacterial antigens, for example the mycobacterial cell wall glycolipid lipoarabinomannan, are also described. Control cells comprising one or more described antigen or fragment thereof (or polynucleotide encoding such a polypeptide) may be useful in methods of detecting and/or isolating mycobacteria, for example in diagnosing tuberculosis, particularly active tuberculosis.

U.S. Pat. No. 5,851,761 (incorporated herein by reference) describes gene probes that can be used to distinguish between M. tuberculosis, M. bovis and BCG strains, and that can also be used to distinguish between different strains of M. tuberculosis. The probes do not show significant hybridisation to nucleic acids from M. paratuberculosis, M. intracellulare, M. scrofulaceum, M. phlei, M. fortuitum, M. kansasii, M. avium, M. malnioense, M. flavescens, M. gordonae and M. chelonei. Thus, control cells comprising one or more such polynucleotide sequences (or polypeptide encoded by such sequence) may be useful in methods of isolating and/or detecting or distinguishing particular mycobacterial strains, as will be apparent to those skilled in the art.

U.S. Pat. No. 5,871,912 (incorporated herein by reference) describes genes and mutations therein that are linked with drug resistance in mycobacteria. The KatG gene, which encodes HPI catalase-peroxidase appears to confer sensitivity to isoniazid. A control cell for a method of characterising mycobacteria in relation to drug resistance may comprise a polynucleotide sequence derived from the KatG gene or variant thereof, in particular a polynucleotide sequence that is linked with isoniazid resistance (for example, a deletion-mutated sequence).

WO99/51748 (incorporated herein by reference) relates to fusion polypeptides comprising at least two M. tuberculosis antigens. It also describes methods of identifying suitable antigens and coding sequences therefor. Control cells comprising one or more M. tuberculosis antigens (or polynucleotide sequences encoding them) described in WO99/51748 may be useful as control cells in methods of isolating and/or identifying mycobacteria, particularly M. tuberculosis.

WO98/53076 (incorporated herein by reference) also relates to antigenic portions of M. tuberculosis proteins, and DNA sequences encoding such polypeptides. Control cells comprising one or more M. tuberculosis antigen (or polynucleotide sequences encoding them) described in WO98/53076 may be useful as control cells in methods of isolating and/or identifying mycobacteria, particularly M. tuberculosis.

U.S. Pat. No. 6,008,201 (incorporated herein by reference) relates to a polypeptide or polypeptides, and polynucleotide sequence encoding them, that confers on M. tuberculosis the ability to enter mammalian cells and to survive within macrophages. WO99/9186 (incorporated herein by reference) relates to polypeptides exported from mycobacteria and polynucleotides encoding them, for example a polypeptide termed DP428 of about 12 kDa. Control cells comprising such a polypeptide or a portion thereof (and/or a polynucleotide sequence encoding the polypeptide or a portion thereof) may be useful in methods of isolating and/or detecting mycobacteria, particularly M. tuberculosis, for example in the diagnosis of tuberculosis.

U.S. Pat. No. 5,928,901 (incorporated herein by reference) relates to peptide sequences corresponding to epitopes characteristic of a protein produced by Plasmodium falciparum in hepatocytes. Molecules comprising such an epitope are used in assays and diagnostic kits for malaria. A control useful in an assay for malaria may comprise such a molecule or epitope.

Control cells may be used in relation to tests relating to non-infected mammalian cells, for example cancer cells or particular cell types of interest. In addition to those indicated above, the following definitions are used in relation to controls for isolation, identification and/or characterisation of mammalian cells.

By a metastatic/malignant cell component is meant a component of a metastatic/malignant cell which is substantially not otherwise expected to be present in other cells in the sample, for example fluid, smears, or tissue sections, from which it is intended to attempt to isolate or identify the metastatic/malignant cell. Examples of such components characteristic of metastatic/malignant cells will be well known to those skilled in the art. Examples of such components are indicated below. As indicated above, cell surface components may be particularly suitable for inclusion in a control cell and as the basis of an isolation procedure. However, non-surface components may also be useful.

Examples of cell surface antigens that may be used as the basis of a method of identifying and/or isolating an appropriate target cell type are indicated in the following tables. A control cell for use in such a method comprises the relevant cell surface antigen, preferably expressed from a recombinant nucleic acid construct.

TABLE 1
Cell surface antigens for targeting

| | | |
|---|---|---|
| Antigen | Antibody | Existing uses |

a) Tumour Associated Antigens

| | | |
|---|---|---|
| Carcino-embryonic | C46 (Amersham) | Imaging and therapy |
| Antigen | 85A12 (Unipath) | of colon/rectum |
| | | tumours. |
| Placental Alkaline | H17E2 (ICRF, | Imaging and therapy |
| Phosphatase | Travers & Bodmer) | of testicular and |
| | | ovarian cancers. |
| Pan Carcinoma | NR-LU-10 (NeoRx | Imaging and therapy |
| | Corporation) | of various carcinomas |
| | | including small cell |
| | | lung cancer. |
| Polymorphic Epithelial | HMFG1 (Taylor- | Imaging and therapy |
| Mucin (Human milk | Papadimitriou, ICRF) | of ovarian cancer and |
| fat globule) | | pleural effusions. |
| β-human Chorionic | W14 | Targeting of |
| Gonadotropin | | carboxypeptidase to |
| | | human xenograft |
| | | choriocarcinoma in |
| | | nude mice (Searle et al |
| | | (1981) Br. J. Cancer |
| | | 44, 137-144). |
| A carbohydrate on | L6 (IgG2a)¹ | Targeting of alkaline |
| Human Carcinomas | | phosphatase (Senter et |
| | | al (1988) PNAS USA |
| | | 85, 4842-4846. |
| CD20 Antigen on B | IF5 (IgG2a)² | Targeting of alkaline |
| Lymphoma (normal | | phosphatase (Senter et |
| and neoplastic) | | al (1988) PNAS USA |
| | | 85, 4842-4846. |

b) Immune Cell Antigens

| | | |
|---|---|---|
| Pan T Lymphocyte | OKT-3 (Ortho) | As anti-rejection |
| Surface Antigen | | therapy for kidney |
| (CD3) | | transplants. |
| B-lymphocyte Surface | RFB4 (Janossy, Royal | Immunotoxin therapy |
| Antigen (CD22) | Free Hospital) | of B cell lymphoma. |
| Pan T lymphocyte | H65 (Bodmer and | Immunotoxin |
| Surface Antigen | Knowles, ICRF; | treatment of acute |
| (CD5) | licensed to Xoma | graft versus host |
| | Corp., USA) | disease, rheumatoid |
| | | arthritis. |

c) Infectious Agent-Related Antigens

| | | |
|---|---|---|
| Mumps virus-related | Anti-mumps | Antibody conjugated |
| | polyclonal antibody | to diphtheria toxin for |
| | | treatment of mumps. |
| Hepatitis B Surface | Anti HBs Ag | Immunotoxin against |
| Antigen | | hepatoma. |

¹Hellström et al (1986) Cancer Res. 46, 3917-3923
²Clarke et al (1985) Proc. Natl. Acad. Sci. USA 82, 1766-1770

Other antigens include α-fetoprotein, Ca-125 and prostate specific antigen.

U.S. Pat. No. 5,688,641 (incorporated herein by reference) describes methods of distinguishing normal cells from cancerous or precancerous cells, in which the expression level of a candidate tumour suppressor gene is measured. A control cell in which the expression level of a candidate tumour suppressor gene described in U.S. Pat. No. 5,688,641 is reduced may be useful in relation to a method as described in U.S. Pat. No. 5,688,641.

General markers for cancer cells may include activated Ras or p53. Mutations linked with malignancy, for example in the P53 gene, may also be useful as markers for cancer cells.

U.S. Pat. No. 4,885,236 (incorporated herein by reference) suggests that differences in nuclear matrix proteins may be useful in distinguishing different cell types. Control cells comprising different nuclear matrix proteins may be useful in relation to assays, for example as described in U.S. Pat. No. 4,885,236.

A control cell comprising the prostate specific membrane antigen (Israeli et al (1994) Cancer Res 54(24), 6306-6310; Israeli et al (1994) Cancer Res 54(7), 1807-1811) may be useful as a control cell in assays for prostate cancer. One or more recombinant polynucleotide comprising a polynucleotide sequence linked with cancer, in particular prostate cancer, may also be included in the control cell.

Methods of preparing or identifying cells useful as control cells in relation to a particular method of isolating, identifying and/or characterising a target cell type will be apparent to those skilled in the art.

Positive control cells may usefully comprise further components (markers) relevant to therapeutic management decisions, for example drug resistance of metastatic cells, oncogene expression and/or mutation patterns, or cell sub-types. For this purpose, control cells desirably comprise the appropriate markers e.g. cell sub-type specific markers, and/or drug resistance markers, and/or oncogene markers. The marker may be a polynucleotide or other molecule, for example a polypeptide. The marker may be detected by methods well known to those skilled in the art, for example using a technique involving antibody binding or nucleic acid hybridisation.

The following outline procedure may be appropriate for a target cell type for which characterising components have not previously been identified (or for a target cell type for which it is desired to identify further characterising components). The procedure (here applied in relation to a mammalian target cell type) comprises the steps of: isolate appropriate mammalian cDNAs; clone into suitable expression vectors; stably express in suitable cell line (preferably from a different species to the target cell type); establish and select clones expressing recombinant component characteristic of the target cell type; prepare/identify antibodies to the characterising component; develop isolation method making use of the selected antibody(s). The process may be repeated until the control cell comprises all the necessary components for isolation (preferably proteins); for identification (preferably proteins); and for diagnosis (preferably nucleic acid (for example DNA and optionally RNA, containing the appropriate the genetic information for the disease of interest). Methods of selecting or preparing clonal cell lines comprising a component characteristic of a particular cell type are well known to those skilled in the art, for example subtraction library techniques, in which the resultant library is depleted in relation to polynucleotide sequences that are transcribed in the cell type of interest and in a contrasting cell type. Methods of analysing expression in individual cells, for example as described in Simone et al (1998) Trends Genet 14(7), 272-276 or U.S. Pat. No. 5,859,699 may be useful in identifying suitable components for forming the basis of a cell identification/isolation/characterisation assay and for inclusion in a control cell for use in such an assay.

A plant-related use that may become more significant in the future due to environmental concerns is testing for genetic modifications (GM) in plants, seeds etc; for example, maize whole grains can be identified as GM positive or negative with appropriate controls. For example, tissue sections from plants, or sections of grain or pollen may be analysed alongside control cells, for example using ill situ techniques. The control cell may comprise a component characteristic of a particular genetically modified organism, for example a component corresponding to the introduced genetic modification of interest. The positive and any negative control cell may conveniently be a bacterial cell in relation to such uses. Other techniques employed in testing for GM contamination, for example PCR on an extract of the product to be tested may only signify gene present or absent not percentage contamination or the nature of the source. Possible contamination may arise from contact with something from which the GM component is normally expressed, for example from non-plant material, for example from human contact or manure, and may also make interpretation of the results difficult, for example if the component is a mammalian gene or portion thereof.

Examples of genetic modifications for which positive control cells may be useful are discussed in the following references: Landridge (2000) Expression of full-length bioactive antimicrobial human lactoferrin in potato plants. Trans Res 9, 71-78; Fischer et al (2000) Antibody production by molecular farming in plants. J Biol Reg & Hom Agents 14, 83-92; Walmsley & Arntzen (2000) Plants for delivery of edible vaccines. Curr Opin Biotech 11, 126-129; Ruggiero et al (2000) Triple helix assembly and processing of human collagen produced in transgenic tobacco plants. FEBS Lett 469, 132-136; Staub et al (2000) High-yield production of a human therapeutic protein in tobacco chloroplasts. Nature Biotech 18, 333-338; Dunwell (2000) Transgenic approaches to crop improvement. J Exp Bot 51, 487-496.

Positive control cells suitable for use in relation to methods of isolating, identifying and/or characterising mammalian cells include cells, preferably mammalian cells, preferably not from the species of interest, that are modified to express one or more target cell components, for example as defined above or further below, ie one or more component characteristic of the target cell in the context in which the method is performed. The component may allow the positive control cell to be used as a control for isolation, identification, sub-typing, identification of disease markers, and/or identification of drug resistance markers. Preferably, identification and isolation markers are components of the cell type of interest that are antigenic; still more preferably proteins, or parts thereof; yet more preferably membrane proteins. A disease marker may be a portion of DNA coding for the mutation (for example deletion) linked with the disease, and/or chromosome of interest, and/or the species-specific normal sequence, or the equivalent portions of mRNA or protein.

Control cells may be useful in relation to intracellular organisms, ie organisms which are phagocytosed by, or invade, cells of the mammalian host, including cells of the immune system, which may preferentially be detected within the cells of the host. Examples include Neisseria sp and Mycobacterium sp. Organisms where part of the life cycle is within or attached to the cells of a mammalian host (for example Mycoplasma sp and Plasmodium falciparum can be detected either in or attached to the host cell or independently of the host cell. Examples of suitable markers are described in the following references: Guibourdenche & Riou (1996) Meningococci throughout the world: phenotypic and molecular markers. Med Mal Infect 26, 389-392; Tondella et al (1994) Ribotyping as an additional molecular marker for studying Neisseria-meningitidis serogroup-B epidemic strains. J Clin Microbiol 32, 2745-2748.

Examples of positive and negative cell surface and intracellular markers for tumours and/or metastatic cells that may be useful as characterising or further components are discussed in the following papers: Proca et al (2000) MOC31 immunoreactivity in primary and metastatic carcinoma of the liver—report of findings and review of other utilized markers. Appl Immunohist Mol Morph 8, 120-125; Kaufmann et al (2000) Uroplakin III is a highly specific and moderately sensitive immunohistochemical marker for primary and metastatic urothelial carcinomas. Am J Clin Pathol 113, 683-687; Maraj et al (2000) Identification of a novel microsatellite marker tightly linked to the KAI-1 gene for predicting prostate cancer progression. Eur Urol 37, 228-233; Kataoka et al (2000) Annexin VII as a novel marker for invasive phenotype of malignant melanoma. Jap J Cancer Res 91, 75-83; Nitta et al (2000) Immunohistochemical study of MUC1 mucin in premalignant oral lesions and oral squamous cell carcinoma—Association with disease progression, mode of invasion, and lymph node metastasis. Cancer 88, 245-254; Elgamul et al (2000) Prostate-specific membrane antigen (PSMA): Current benefits and future value. Sem Surg Oncol 18, 10-16; Hubert et al (2000) A prostate-specific cell-surface antigen highly expressed in human prostate tumors. PNAS 96, 14523-14528; Millon et al (1999) Detection of prostate-specific antigen- or prostate-specific membrane antigen-positive circulating cells in prostatic cancer patients: clinical implications. Eur Urol 36, 278-285; De Marzo et al (1999) E-cadhering expression as a marker of tumor aggressiveness in routinely processed radical prostatectomy specimens. Urol 53, 707-713; Hamasaki et al (1999) GT1b in human metastatic brain tumours: GT1b as a brain metastatis-associated ganglioside. Biochem Biophys Act Mol Cell Biol Lipid 1437, 93-99; Gnirke & Weidle (1999) Investigation of prevalence and regulation of expression of Progression Associated Protein (PAP). Anticancer Res 18, 4363-4369; Raikhlin et al (1997) Membrane proteases in human malignant lymphomas. Acta Hist Cytochem 30, 513-516; Chao et al (1996) Expression of Epstein-Barr virus-encoded RNAs as a marker for metastatic undifferentiated nasopharyngeal carcinoma. Cancer 78, 24-29; Schadendorf et al (1995) Membrane-transport proteins associated with drug-resistance expressed in human-melanoma. Am J Pathol 147, 1545-1552; Puhlmann et al (1994) Lack of expression of a 31/33 kD surface protein on human colon-carcinoma cells is a marker for metastasizing potential. Anticancer Res 14, 2701-2707; Josefsson et al (2000) Viral load of human papilloma virus 16 as a determinant for development of cervical carcinoma in situ: a nested case-control study. Lancet 355, 2189-2193; Kannan et al (2000) FHIT Gene mutations and single nucleotide polymorphism in Indian oral and cervical squamous cell carcinomas. Oral Oncol 36, 189-193; Vambutas et al (2000) Altered expression of TAP-1 and major histocompatibility complex class I in laryngeal papillomatosis: correlation of TAP-1 with disease. Clin Diag Lab Immunol 7, 79-85.

Examples of recombinant cells suitable for use as control cells in relation to higher organism, for example mammalian cell testing are indicated in the following table. It will be appreciated that the other combinations of control cell background, characterising and further components are also suitable as control cells. Components indicated as characterising components may also be used as confirmatory components (or vice versa), such that a control cell may have two or more components indicated in the table as characterising components. Interaction of a target cell selective reagent with the characterising component may be used in identifying, characterising and/or isolating the target or positive control cell.

| Target cell | Characterising | Further | Further | Control cell |
|---|---|---|---|---|
| | component | (confirmatory) | (diagnostic) | background |
| | | component | component | |
| Prostate | Prostate | PSA (prostate | Tumour | CHO cell |
| cancer cell | specific | specific | suppressor | |
| | membrane | antigen) | gene (see U.S. Pat. No. | |
| | antigen (Isreali | | 5,688,641) | |
| | et al (1994)) | | | |
| Mycobacterially- | 40 kDa | 19 kDa | KatG gene | CHO cell |
| infected | antigen (see | lipoprotein | sequence | |
| cell | WO98/36089) | (see | (drug | |
| | | WO98/04711) | resistance) | |
| | | | (see U.S. Pat. No. | |
| | | | 5,871,912) | |
| Hepatitis B- | Hepatitis B | Hepatitis B | Strain-specific | CHO cells |
| infected cell | surface antigen | intracellular | nucleic acid | |
| | | antigen | sequence | |
| Viral | Portion of | | MUC1 mucin | Saccharomyces |
| induced | Epstein Barr | | expression | |
| nasopharyngeal | viral RNA | | | |
| tumour | | | | |
| cell | | | | |
| Viral | Papilloma | Genetic | Decreased | H4IIe cells |
| induced | Viral DNA | alterations at | TAP-1 levels | |
| cervical | | the FHIS | | |
| tumour cell | | (fragile | | |
| | | histidine triad) | | |
| | | tumour | | |
| | | suppressor | | |
| | | gene | | |
| Neisseria sp | Outer | Ribotyping | A portion or | HepG2 |
| | membrane | analysis for | product of an | |
| | protein | rRNA gene | antibiotic | |
| | | restriction | resistance | |
| | | pattern (Rb1) | gene/genes of | |
| | | | specific | |
| | | | clinical | |
| | | | interest, eg | |
| | | | penicillin | |
| | | | resistance | |
| Hepatoma | Alpha | Loss of | Multidrug | Cos 7 |
| cell | fetoprotein | glucose-6- | resistance | |
| | | phosphatase | gene | |
| | | | expression | |
| | | | pattern | |

Role of the Control Cells

A control cell may be used as one or more of the following types of control.

As noted above, the positive control cell may serve as a control for all cells of a particular classification group. For example, the positive control cell may serve as a positive control for all bacterial cell types (in which case it has a marker molecule present in all (which includes the meaning of substantially all) bacterial cell types), or alternatively as a control for (for example) all streptococci (in which case it has a marker molecule present in all (which includes the meaning of substantially all) streptococcal strains but not necessarily in other bacterial cells). Preferably, the positive control cell comprises a marker molecule present in all (or substantially all) cells of a particular classification group (target cells), but not present in other cells falling outside that group but falling within the next highest level classification grouping. Thus, in the example given above a preferred positive control cell for streptococci comprises a marker that is present in all streptococcal strains but that is not present in non-streptococcal bacteria. The preferred positive control cell may comprise further markers, for example a marker that is present in all bacterial cell types and/or a marker that is present only in particular streptococcal strains.

The control cell may be suitable for use as a control for cellular isolation procedures and/or for identification/characterisation procedures. If the cell is to be used as a control for an isolation procedures then it comprises a molecule (characterising component) which is present in the cell type to be isolated and whose presence is necessary and sufficient for a cell to be isolated using the isolation procedure. Preferably the molecule is a cell-surface molecule. Still more preferably, the molecule reacts specifically with an antibody which is used as a means of conferring specificity for the desired cell type on the isolation procedure.

If the cell is to be used as a control only for identification/characterisation procedures (for example as a control in assays which do not involve a cell isolation step, or as a control for steps in an assay that follow any cell isolation step) then it is not necessary for the cell to comprise a recombinant molecule which allows it to be isolated in the same way as a target cell. In such a case the control cell may comprise a (preferably recombinant) molecule that is specific for the target cell type but whose presence does not have to be necessary and sufficient for the cell to be isolated using any isolation procedure. For example, the molecule may be a recombinant polynucleotide or intracellular polypeptide which need not confer a recognisable phenotype on the cell. However, the molecule must be suitable for specific detection, for example following lysis of the cell. Thus, for example, the recombinant polynucleotide may be detected using hybridisation-based techniques well known to those skilled in the art.

Control cells as discussed above may be useful when performing an isolation/identification/characterisation method, which may be termed performing an assay. It will be appreciated that a control cell sample may be analysed on each occasion on which a test sample is analysed; or when setting up an assay procedure, followed by optional analysis of further control cell samples, for example on a periodic basis. The characteristics of each assay will determine the optimum frequency of analysis of control cell samples, as can readily be determined by those skilled in the art. When performing clinical diagnostic assays, it may be appropriate to analyse a positive (and preferably also a negative) control cell sample in parallel with each test sample. If several test samples are analysed simultaneously, a separate positive or negative control cell sample may not be required in relation to each test sample; the characteristics of the assay and the circumstances under which it is performed will determine how many control cell analyses are required. For example, for assays performed individually in a home or GP's surgery setting, for example using a disposable test device, a cellular control may be incorporated in each test device such that the cellular control is analysed alongside the test sample. Of course, both positive and negative controls may be incorporated in each test device. Further, several different types of positive (and optionally negative) control cells may be included in each test device, preferably corresponding to the number of different target cell selective reagents which are intended to be used with the test device.

Control cells as discussed above may also be useful when optimising or validating an assay procedure or assay reagents. They may also be useful for checking reagents for use in such an assay ie for carrying out quality control tests.

A further aspect of the invention provides a method of testing a procedure for identifying and/or characterising a target cell in, and/or isolating a target cell from, a sample which may contain said target cell, comprising the steps of

1) providing a target cell selective reagent which interacts with a characterising component derivable from the target cell,

2) providing a positive control sample comprising a positive control cell, wherein the positive control cell comprises said characterising component, is preferably recombinant, and is not identical to the target cell,

3) identifying and/or characterising the positive control cell in, and/or isolating the positive control cell from, the positive control sample by performing on the sample a process involving interaction of the target cell selective reagent with the characterising component of the positive control cell,

wherein the method for identifying, isolating and/or characterising a selected type of cell is considered to be satisfactory if the positive control cell is identified, isolated and/or characterised correctly in step 3.

The method may involve further steps in which the specificity of the target cell selective reagent for the target cell type is further investigated. For example, the test procedure may be repeated using non-target/non-target control cells (for example negative control cells, as discussed above) in order to determine whether non-target cells will also be isolated, identified and/or characterised using the assay procedure; if so, changes in the target cell selective reagent or the assay procedure may be required in order to improve specificity. Steps involved in optimising an assay procedure will be well known to those skilled in the art; the method of the present invention provides the use of a positive control cell (which is preferably recombinant, as noted above) in place of a target cell when performing the optimisation tests.

The equivalence of the positive control cell with the target cell with respect to the components of interest may be established by comparison of the target cell with the positive control cell. For example, Streptococcus target cells may be grown in culture by standard methods and thereafter fixed onto a microscope slide adjacent to positive and/or negative control cells. The slide may then be analysed by immunohistochemistry and/or molecular biology techniques. If both target cell and positive control cell give a positive reaction then the positive control cell may be considered to be equivalent with the target cell and suitable for use as a positive control cell in relation to that target cell. The negative control cell may be suitable for use as a negative control cell in relation to that target cell if it does not give a positive reaction.

Control cells may be used in an assay system as internal limits for detection, ie high and low, as well as acting as quality control limits for comparison with other laboratories as well as controls for different assay runs within the same laboratory. Similarly, negative control cells may be used as indicators of non-specific reactivity levels.

The quality control limits can be set using control cells expressing a single level of (for example) a protein of interest. If the detection system is adjusted, for example by altering the level of primary antibody then the signal detected is proportional to the level of added primary antibody (see Histol Histopathol (1995) 10, 979-999). Alternatively the limits can be set by using a number of different control cells, each expressing a different level of the protein of interest.

Control cells can be used to optimise each individual component of the assay, for example the optimum temperature for hybridisation of a nucleic acid probe. Similarly, the optimal antibody titre can be established using control cells. Control cells may also be used to optimise fixation methods, and the detection systems of image analysis auto-analysers may also be calibrated using control cells.

The use of cellular controls according to the present invention may also allow automation, improved quality control and reliability, reduced dependence on histopathologist expertise (and hence reduced costs and potentially faster assay performance) of pathological diagnosis (for example of inflammation, infection, developmental disorders, malignant change, abnormal metabolic processes, degenerative changes or forensic analysis, in human or other species) in solid tissues and in cellular samples, for example cervical smears, solid tissue samples (both frozen and fixed), blood smears, cells in samples from the urogenital, respiratory, gastrointestinal tracts by aspirates, smears and brushings, and pus. Target cells may be in so high a concentration that isolation methods are not required; in some cases the sample may be a semi-solid, particularly at room temperature. Cells may also be taken from dividing embryos in relation to in vitro fertilisation. These cells are required for the diagnosis of genetic and chromosomal disorders in humans, and in addition in animals for the selection of desired traits.

Positive control cells may be used as positive controls for a diagnostic process that uses microscopy to identify individual cells of interest, for example using a slide based technology (for example a tissue section or cell smear). Subsequent to the identification of the cell of interest, the determination of further relevant characteristics of the cell, preferably therapeutically significant characteristics, for example performing diagnostic test(s), can be slide or tube based.

As will be apparent to those skilled in the art, methods for isolating, identifying and/or characterising cells may be performed on a range of samples. For example, tests for pathogens may be performed on environmental or host liquid samples, or on host tissue samples.

Environmental samples include samples from water (pipes, tanks, bodies of water, rivers, lakes, reservoirs, sea, sewage plants and effluent discharges), soil and air, including air filtering devices. The samples may be tested in relation to, for example, algae (including toxic blooms in reservoirs), pollen, dust mites, Cryptosporidium sp, Legionnaires sp, Mycobacterium sp (for example in air filters, soil, milk etc), bacterial spores, fish or crustacean parasites (as discussed above), Q fever (Coxiella burnetti) in bone meal or hides.

Diagnostic tests in relation to cells from mammals, for example humans or domestic animals, may be performed on fluid samples that may contain cells in normal and/or diseased states. The sample may preferably be derived from the blood circulation (including serum and plasma), cerebrospinal fluid circulation, lymphatic circulation, synovial fluid, tears, pleural, peritoneal, pericardial, gastrointestinal tract, biliary system, urinary tract, semen, cervical or vaginal fluids, vitreous or aqueous humour, milk, fluid from cysts (for example ovarian), sputum or amniotic fluid.

In the routine clinical diagnostic laboratory, isolation of very small numbers of, for example, fetal or metastatic cells from bodily fluids containing relatively large numbers of other cell types requires a positive control. Inclusion of the positive control cell makes it possible to differentiate between a failure of the technique, and the absence of the cell of interest. A positive control sample may consist of a few of the relevant recombinant control cells in a fluid, which may also contain large numbers of the parent (non-engineered) cell (negative control cell) from which the control cell was derived. The efficacy of the isolation technique may be determined if such a control sample is used.

For the manufacturer to design, to optimise, and to control the quality of the isolation and/or diagnostic kit(s), for example where very small numbers of fetal or metastatic cells are being recovered from bodily fluids containing relatively large numbers of other cell types, requires a readily available standard control. A standard control may consist a few of the relevant positive control cells in a fluid or on a slide, as appropriate for the assay design, which preferably also contains large numbers of the parent (non-engineered) cell from which the control cell was derived (negative control cells). The ratio of positive to negative control cells is not critical.

Preferably, the positive control cells may contain a marker of the target cell that is different to the characteristic used for isolation, ie a further (confirmatory) component found in the target cell. Preferably, the parent non-engineered cells (or other negative control cell type) do not contain the confirmation/identification, nor the isolation (characterising) component. Controls with different levels of expression of the confirmation/identification, or the characterising (isolation) component, may allow detection limits to be determined.

Use of a positive (and optionally negative) control cell according to the present invention may be particularly important when performing assays, for example assays directed towards diagnosis of a specific disease, using one or more individual cells. The control cell contains, for example, the mutation of interest and/or the relevant normal sequence for the normal target cell type. Methods of genetic analysis are well known to those skilled in the art. Exemplary methods are described, for example, in WO98/40746.

Methods by which single cells may be analysed include methods in which the technique of Laser Capture Microdissection (LCM) is used. This technique may be used to collect single cells or homogeneous cell populations for molecular analysis and is described in, for example, Jin et al (1999) Lab Invest 79(4), 511-512; Simone et al (1998) Trends Genet 14(7), 272-276; Luo et al (1999) Nature Med 5(1), 117-122; Arcuturs Updates, for example June 1999 and February 1999; U.S. Pat. No. 5,859,699 (all incorporated herein by reference). The cells of interest are visualised, for example by immunohistochemical techniques, and transferred to a polymer film that is activated by laser pulses. The technique may also be used for isolation of cells which are negative for a particular component. Microscopes useful in performing LCM are manufactured by Arcturus Engineering, Inc., 1220 Terra Bella Avenue, Mountain View, Calif. 94042, USA.

LCM may be used with other isolation or detection methods. For example, LCM may be used following an isolation method which enriches the sample for the target cell type. A positive control cell may have the component used as the basis for the isolation (including enrichment) or detection method and/or the component (which may be a different component or the same component) used for the LCM step.

LCM may be particularly useful in the analysis of fetal cells, for example obtained from maternal blood.

Control cells may help in the development of non-invasive diagnostic and/or treatment regimens by helping in identification of specific defects in metastatic tumour cells isolated from body fluids, obviating the need for invasive biopsy. Control cells may help in developing a comprehensive and accurate body of data concerning the correlation between particular characteristics, for example changes resulting in drug resistance; inherited and spontaneously arising mutations; changes in patterns of expression of key proteins such as oncogenes, and particular cancers or therapeutic outcomes.

The invention will now be described in more detail by reference to the following, non-limiting Figures and examples.

FIG. 1: Diagrammatic representation of target and corresponding control cell. The target cell selective reagent interacts with a characterising component of the target cell which is also present in the recombinant control cell. This interaction may be used in isolating the target cell from, and/or identifying or characterising the target cell in, the test sample. The further (confirmatory) reagent may be used to confirm that the intended cell type has been isolated, identified and/or characterised, or to further characterise the cell. The further (confirmatory) reagent may interact with a cell surface or intracellular component. The polynucleotide hybridising to a genomic sequence of interest may be, for example, a polynucleotide hybridising to a gene conferring drug resistance (for example if the target cell is a bacterial cell or cancer cell), or a polynucleotide hybridising to a sequence linked with an inheritable disease or condition, for example cystic fibrosis. In the latter case, the target cell may be a fetal cell, which may be obtained from maternal blood. The target cell selective reagent and further (confirmatory) reagent may interact with fetal cell components, for example human adult liver cell components, which are found in fetal cells but not in other cells present in maternal blood.

FIG. 2: Diagrammatic representation of an assay in which the positive control cell of FIG. 1 and corresponding negative control cell are used. The characterising component is used in isolating the test and control cells. The confirmatory component is used in confirming that the intended cell type has been isolated, using a labelled reagent that binds specifically to the confirmatory component. The polynucleotide hybridising to a genomic sequence of interest is used in genetic analysis of the isolated target cell.

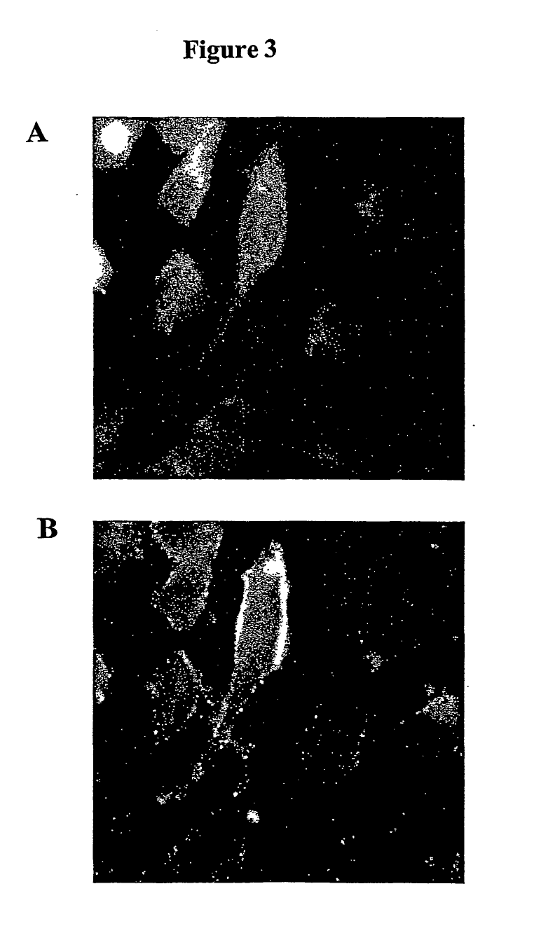

FIG. 3: Immunofluorescent images of a mixture of V79 cells to demonstrate differential transfection of cells. Cells in the top left quarter of the image are doubly transfected with UGT1AI and MRP1. Magnification: x1140.

A) Immunofluorescent image using the primary antibody anti-UGT1A1 and fluorescein-labelled secondary antibody and visualised using an Openlab, Improvision Image Analysis System. UGT1A1 is an endoplasmic reticulum membrane component and the pattern of green colour throughout the cell cytoplasm is consistent with an endoplasmic reticulum membrane location.

B) Immunofluorescent image using the primary antibody anti-MRP1 and rhodamine-labelled secondary antibody and visualised using an Openlab, Improvision Image Analysis System. MRP is a plasma membrane component and the intense red colour around the periphery of the cells is consistent with a plasma membrane location.

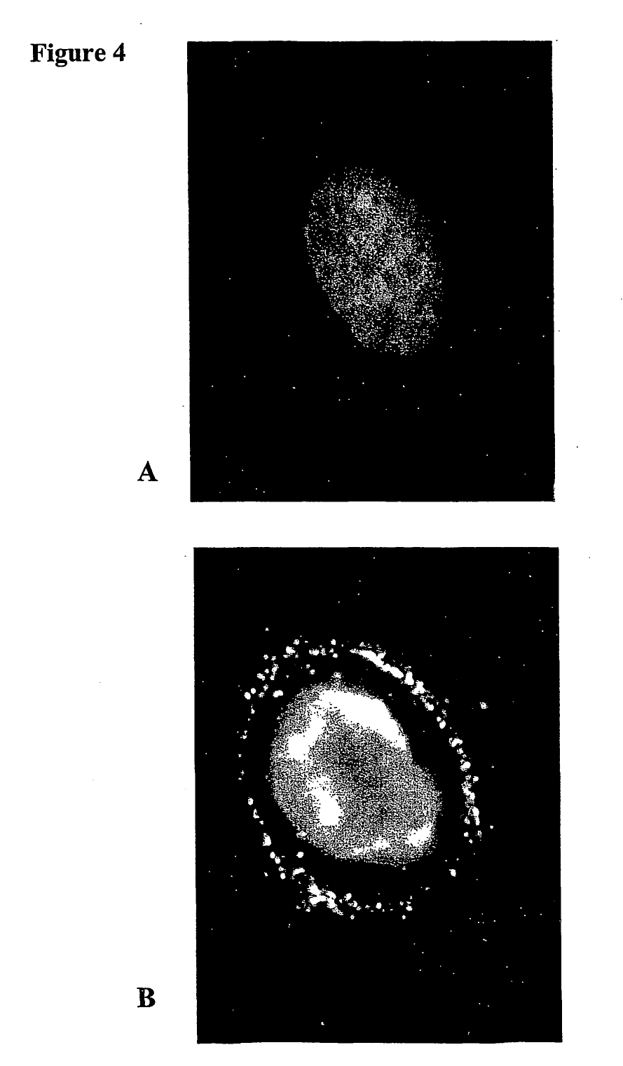

FIG. 4: Mammalian cell lines incubated with both DAPI and a primary antibody to human GLUT2 followed by secondary FITC labelled antibody. A) A negative control cell (untransfected CHO-K1 cell) showing a DAPI fluorescent positive image outlining the nucleus but negative for FITC staining. B) A positive control cell expressing GLUT2 (see Example 3) showing a DAPI fluorescent image outlining the nucleus with positive FITC staining of the plasma membrane consistent with the plasma membrane location of human hepatic GLUT2 protein.

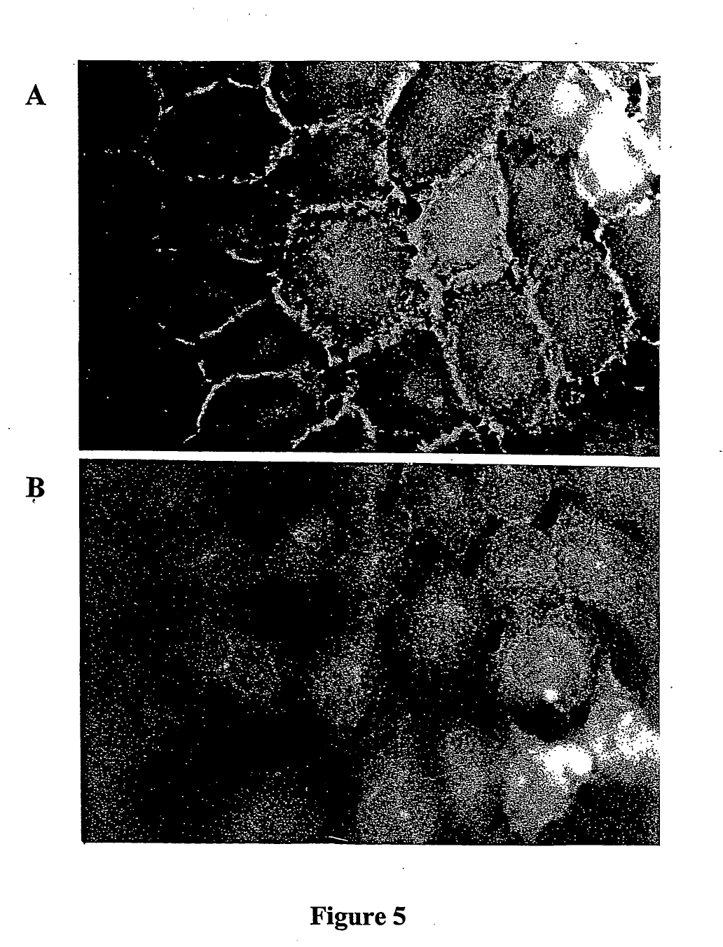

FIG. 5: Control cells used to calculate efficiency of purification procedures using antibodies to antigens expressed in positive control cells and test cells of interest.

Photomicrograph showing results of purification procedures described in Example 4. A) The eluate contains cells with a positive intense red fluorescence around the periphery of the majority of the cells; B) in contrast the majority of the cells in the flow-through cell fraction do not show the intense staining at the periphery. The peripheral staining is consistent with the plasma membrane location of the test antigen MRP1.

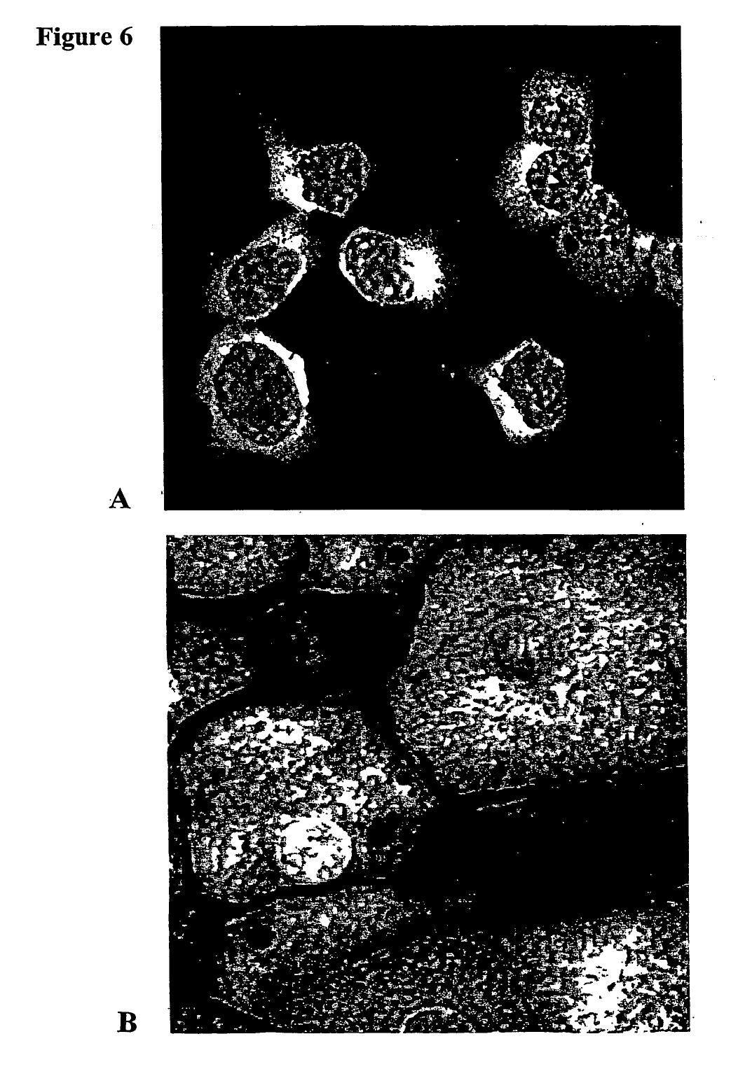

FIG. 6: Control cells as positive and negative controls for immunohistochemistry in solid tissues

A) Transfected mammalian cells (positive control cells) expressing glucose-6-phosphatase showing immunoreactivity to endoplasmic reticulum glucose-6-phosphatase after incubation with a primary sheep antibody monospecific for hepatic microsomal glucose-6-phosphatase enzyme and a FITC labelled secondary antibody; B) a adult human liver slice showing immunoreactivity to endoplasmic reticulum glucose-6-phosphatase after incubation with a primary sheep antibody monospecific for hepatic microsomal glucose-6-phosphatase enzyme.

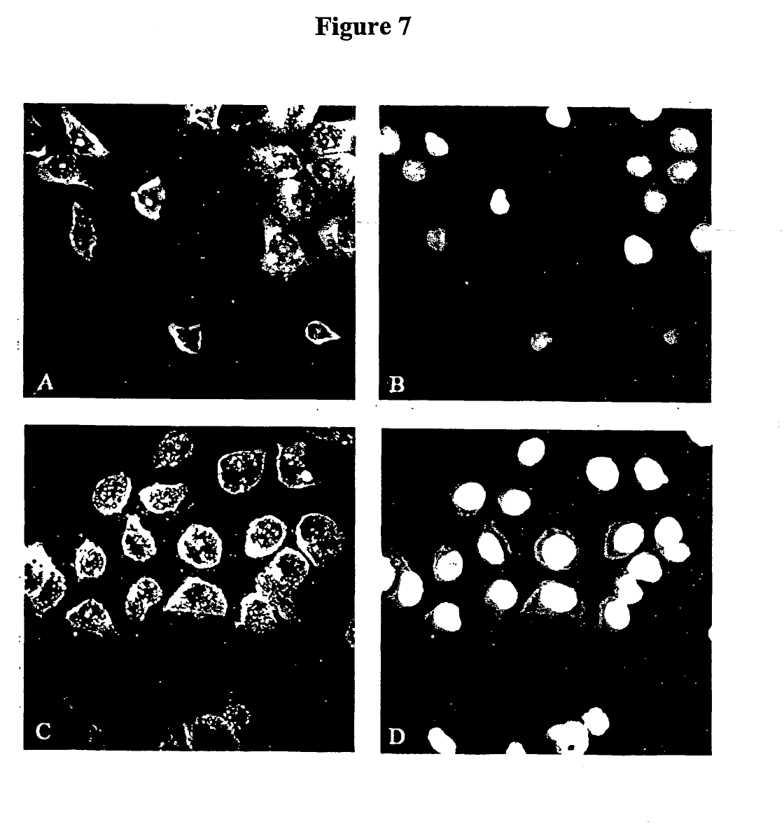

FIG. 7: Positive and negative control cells for optimisation and quality control of cell enrichment/purification procedures

GLUT2 was cloned into a mammalian expression vector, stably transfected into H4Iie to make positive control cells and selected using the antibiotic zeocin. The photomicrograph shows an example of a mammalian cell line (H4IIe) incubated with DAPI, and a primary antibody to human GLUT2 followed by a secondary FITC labelled antibody. Panels B) and D) show DAPI fluorescence. Panels A) and C) show FITC fluorescence.

Panels A) and B) represent a mixture of positive and negative control cells. Positive cells show plasma membrane fluorescence to GLUT2. Panels C) and D) are the same mixture of positive and negative control cells following incubation of cells with GLUT2 antibody and subsequent isolation of GLUT2 positive cells using secondary antibody attached to a solid support.

### EXAMPLE 1

### Control Cells for Fetal Cell Isolation/Identification

V79 cells (Chinese hamster lung cells) expressing UDPGT1A1 (an internal endoplasmic reticulum marker) and multi-drug resistance (MDR) protein (a plasma membrane marker) were constructed.

The cells further comprise a polynucleotide with a mutation found in hypercholesterolaemia.

The control cells were prepared as follows. Uridine diphosphate glucuronosyltransferase (UGT) 1AI was cloned into pcDNAneo1 and stably transfected into V79 cells. MRP1 was cloned into pcDNA3.1+zeo and stably transfected into the V79 cells. MRP1 in pcDNA3.1+zeo was also stably transfected into V79 cells expressing UGT 1AI. Four types of cells were thereby generated:

1) V79-negative control cells

2) V79 plus (UGT)1AI control cells for MRP1 but positive controls for (UGT)1AI

3) V79 plus MRP1 negative control cells for (UGT)1AI but positive controls for MRP1

4) V79 plus MRP1 and (UGT)1AI double positive control cells.

The control cell may alternatively or in addition comprise other plasma membrane protein components, for example GLUT 2 or other plasma membrane protein, as described in WO98/40746, or a CD protein (ie a Cluster of Differentiation protein, commonly expressed on cell surfaces, as known to those skilled in the art). These are expressed stably using standard molecular biology techniques in suitable cells. The cells are preferably mammalian cell lines, preferably cells lines from a species other than the species of interest, preferably with relatively low adhesive properties to laboratory ware in which they grown, and preferably cells which do not normally express the marker of choice (as for some purposes the untransfected cells may be used as a negative control). CHO (Chinese hamster ovary) cells may have suitable low adhesion and may be suitable as a parent line for preparing a control cell for use in relation to human cell types.

The control cell may alternatively or in addition comprise other markers useful in checking the correct identification/isolation of the target cells, for example for testing the efficacy of identification antibodies. Suitable markers include liver endoplasmic reticulum proteins and/or any other internal protein including specific epitopes, for example as described in WO98/40746, or as identified using studies on single cells, for example as reviewed in Simone et al (1998) Trends Genet 14(7), 272-276 and described in U.S. Pat. No. 5,859,699, or using chip technology or global amplification strategy to determine gene expression patterns in small numbers of cells (e.g. Brady et al (1995) Analysis of gene-expression in a complex differentiation hierarchy by global amplification of cDNA from single cells. Curr Biol 5(8), 909-922).

The control cells may further comprise recombinant polynucleotides representing a gene or part thereof, or cDNA or part thereof, having a sequence which correlates with either the presence or absence of a particular disease. A sequence that may be useful in distinguishing a Y from an X chromosomes may be useful to include as a control for sex determination. Sequences characteristic of particular chromosomes (or chromosome break point) may also be useful to include.

### EXAMPLE 2

### Assays using Control Cells

Bacterial Diagnosis in the Clinic: 1

An HIV positive adult presents with a cough but insignificant sputum. He is clearly unwell with pyrexia and vomiting. The chest X-ray shows some pneumonic changes. He has failed to respond to a course of broad spectrum antibiotics including erythromycin and co-trimoxazole. His condition is deteriorating. One of the likely possibilities is reactivation of Mycobacterium tuberculosis for which he has previously been treated, and that he has a multi-drug resistant strain. A lung biopsy is taken and sections are immunostained alongside positive CHO control cells expressing 40 kDa antigen, 19 kDa lipoprotein and the Kat G gene product and negative control CHO cells to allow identification of M. tuberculosis and its drug resistance on the same day. The alternative identification of Mycobacterium and its drug resistance is the traditional histochemical stain for acid and alcohol fast bacilli and culture to determine resistance patterns, which may take up to three months.

Bacterial Diagnosis in the Clinic: 2

A 750 g 26 gestation premature infant is being ventilated in a neonatal intensive care unit and the secretions from the endotracheal tube become increasingly purulent in spite of treatment with penicillin and gentamicin. The infant's condition is deteriorating rapidly. Purulent secretions are smeared onto a glass slide and fixed and immunostained alongside positive yeast control cells expressing Outer membrane protein H1 and portions of several different products of antibiotic resistance genes of specific clinical interest, eg Gentamicin, Tobramycin, Ciprofloxacin, Cephotaxime etc, and negative yeast control cells. The results show the presence of Pseudomonas sp and the antibiotic sensitivity pattern within one day. The alternative is conventional culture and determination of drug sensitivities which normally will take 48-72 h. Such a delay has the risk of increasing morbidity and mortality.

Fetal Cell Testing in the Clinic

Mrs A, who is 44 years old, is pregnant. In view of her age, early diagnostic fetal ultrasound was performed at an early stage in pregnancy, which suggested increased nuchal lucency, often associated with Down's syndrome (Trisomy 21). A maternal blood sample was taken, and fetal red blood cells were separated from maternal blood cells by methods described in WO98/40746. The isolated cells were subjected to conventional immunohistochemistry and were then scanned by an automated image scanning device which used V79 cells expressing UDPGT1A1 as a positive control to allow confirmation of origin. In situ hybridisation techniques showed that fetal cells were trisomic for chromosome 21. The alternative would have been the use of established invasive methods such as chorionic villus biopsy or amniocentesis, which would have inherent risks, to obtain fetal cells.

Metastatic Cell Isolation Kit Testing in a Diagnostic Company

A diagnostic firm wishes to develop a kit to isolate and characterise circulating tumour cells from the blood of men with metastatic tumours and initially to develop a kit based on prostate cancer. They developed the kit using engineered control cells; CHO cells expressing prostate specific membrane antigen. The kit is optimised for isolation by passing mixtures of “wild type” CHO cells and prostate specific membrane antigen positive CHO cells through a magnetic cell activated sorting selection system using antibodies to prostate specific membrane antigen.

Metastatic Cell Isolation and Identification in the Clinic

Mr D is 55 years old and was noticed to be jaundiced. A liver ultrasound scan showed the presence of multiple lesions consistent with metastatic tumour deposits. It is clinically useful if tumour cells can then be characterised, both in terms of origin, and in terms of drug susceptibility, without having invasive investigation with biopsy sampling. Using the kit developed by the diagnostic firm (see above) it was shown that metastatic tumour cells were isolated from Mr D's blood, and these were shown to be of prostate origin by immunohistochemistry using CHO cells expressing prostate specific antigen as a positive control.

### EXAMPLE 3

### Control Cells as Positive and Negative Controls for Isolation and Identification of Human Embryonic and Fetal Nucleated Red Blood Cells

GLUT2 was cloned into a mammalian expression vector, stably transfected using Lipfectin into CHO-K1 cells grown in low glucose media, and selected using the antibiotic neomycin, to make positive control cells. Photomicrograph FIG. 4 shows an example of a mammalian cell line (CHO-K1) incubated with both DAPI, and a primary antibody to human GLUT2 followed by a secondary FITC labelled antibody: A) a negative control cell (un-transfected CHO cell) showing a DAPI fluorescent positive image outlining the nucleus but negative for FITC staining; B) a positive control cell showing a DAPI fluorescent image outlining the nucleus with positive FITC staining of the plasma membrane consistent with the plasma membrane location of human hepatic GLUT2 protein. Such cells are excellent positive and negative controls for isolation and identification of human embryonic and fetal nucleated red blood cells, which express GLUT2 immunopositivity.

### EXAMPLE 4

### Control Cells used to Calculate Efficiency of Purification Procedures using Antibodies to Antigens Expressed in Positive Control Cells and Test Cells of Interest

MRP1 was cloned into a mammalian expression vector and stably transfected into V79 cells to make positive control cells and selected using the antibiotic zeocin. Two types of cells were used in the experiment, untransfected V79 cells were used as negative controls, and V79 cells expressing MRP1 were used as positive control cells. The cells were mixed and incubated for one hour with anti Multidrug Resistance-Related Protein (MRP) antibodies. IgG microbeads and a Magnetic Cell Sorting System (MACS) were used to isolate and enrich the V79 cells expressing MRP1 from the cell mixture. The flow-through cell fraction largely contained the negative controls untransfected V79 cells. The eluate largely contained the positive V79 control cells expressing MRP. Both the flow through fraction and the eluate were dilute and hence concentrated by centrifugation onto glass microscope slides using a Cytospin. The concentrated cells were visualised using a rhodamine-labelled antibody and an Openlab Improvision Image analysis System. The photomicrographs of FIG. 5 shows an example of cells mixed in suspension at a ratio of 10:1 (negative:positive) prior to the procedures described above, A) the eluate containing cells with a positive intense red fluorescence around the periphery of the majority of the cells; B) in contrast the majority of the cells in the flow-through cell fraction did not show the intense staining at the periphery. The peripheral staining is consistent with the plasma membrane location of MRP1. Experiments similar to the ones above in which positive and negative cells are counted before and after purification steps can be used to establish the efficiency and efficacy of cellular enrichment and purification methods.

### EXAMPLE 5

### Control Cells as Positive and Negative Controls for Immunohistochemistry in Solid Tissues

Standard immunohistochemistry methods are traditionally based on visualisation of an end reaction product, usually coloured. Failure of the method often cannot be distinguished from a negative result. Intensity of colours can vary from day to day making direct comparisons and/or automation of techniques difficult. This problem can be overcome with cellular controls in each immunohistochemical run which express standard amounts of the antigen of interest in the appropriate sub-cellular compartment or organelle. FIG. 6 shows: A) transfected mammalian cells (positive control cells) expressing glucose-6-phosphatase showing immunoreactivity to endoplasmic reticulum glucose-6-phosphatase after incubation with a primary sheep antibody monospecific for hepatic microsomal glucose-6-phosphatase enzyme and a FITC labelled secondary antibody; B) a adult human liver slice showing immunoreactivity to endoplasmic reticulum glucose-6-phosphatase after incubation with a primary sheep antibody monospecific for hepatic microsomal glucose-6-phosphatase enzyme.

### EXAMPLE 6

### Positive and Negative Control Cells for Optimisation and Quality Control of Cell Enrichment/Purification Procedures

GLUT2 was cloned into a mammalian expression vector, stably transfected into H4Iie to make positive control cells and selected using the antibiotic zeocin. Photomicrograph FIG. 7 shows an example of a mammalian cell line (H4IIe) incubated with DAPI, and a primary antibody to human GLUT2 followed by a secondary FITC labelled antibody. Panels B) and D) show DAPI fluorescence. Panels A) and C) show FITC fluorescence.

Panels A) and B) represent a mixture of positive and negative control cells. Positive cells show plasma membrane fluorescence to GLUT2. Panels C) and D) are the same mixture of positive and negative control cells following incubation of cells with GLUT2 antibody and subsequent isolation of GLUT2 positive cells using secondary antibody attached to a solid support. This procedure leads to an enrichment/purification of positive control cells compared to the original mixture. This use of control cells allow potential enrichment/purification procedures to be tested and optimised prior to isolation of cells of interest from biological fluids. Such cells can also be used as quality controls of kits to design to isolate cells of interest from fluids.

1. A method for identifying and/or characterising a target cell in, and/or isolating a target cell from, a test sample which may contain said target cell, comprising the steps of
1) identifying and/or characterising any target cell in, and/or isolating any target cell from, the test sample by performing on the test sample a process involving interaction of a target cell selective reagent with a characterising component of the target cell,
2) providing a positive control sample comprising a positive control cell, wherein the positive control cell comprises said characterising component and is not identical to the target cell,
3) identifying and/or characterising the positive control cell in, and/or isolating the positive control cell from, the positive control sample by a process according to step 1 performed on the positive control sample, wherein the method for identifying, isolating and/or characterising the target cell is considered to have been performed correctly if the positive control cell is identified, isolated and/or characterised correctly in step 3.

2. A method for determining whether a method for identifying and/or characterising a target cell in, and/or isolating a target cell from, a test sample which may contain said target cell has been performed correctly, comprising the steps of
1) identifying and/or characterising any target cell in, and/or isolating any target cell from, the test sample by performing on the test sample a process involving interaction of a test cell selective reagent with a characterising component of the target cell,
2) providing a positive control sample comprising a positive control cell, wherein the positive control cell comprises said characterising component and is not identical to the target cell,
3) identifying and/or characterising the positive control cell in, and/or isolating the positive control cell from, the positive control sample by a process according to step 1 performed on the positive control sample,
4) determining whether the positive control cell is identified, isolated and/or characterised correctly in step 3.

3. Use of a positive control cell in a method for identifying and/or characterising a target cell in, and/or isolating a target cell from, a test sample which may contain said target cell, wherein any target cell in the test sample is identified, isolated and/or characterised by a process involving interaction of a target cell selective reagent with a characterising component of the target cell, wherein the positive control cell comprises said characterising component and is not identical to the target cell.

4. The method of claim 1 or 2 or use of claim 3 wherein the step of identifying and/or characterising any target cell in, and/or isolating any target cell from, the test sample further comprises the step of performing on the test sample or on any target cell isolated from said test sample a process involving interaction of one or more further reagents with one or more further components of the target cell,
and the positive control cell further comprises said further component(s).

5. The method or use of any of the preceding claims wherein the method for identifying and/or characterising a target cell in, and/or isolating a target cell from, a test sample which may contain said target cell further comprises the steps of providing a negative control sample comprising a negative control cell but not a target cell or positive control cell, wherein the negative control cell does not comprise said characterising component; performing the process involving interaction of a test cell selective reagent with a characterising component of the target cell on the negative control sample; and determining whether any cell is isolated from, and/or identified and/or characterised in, the negative control sample; or determining the background (negative) result of the process when performed on the negative control sample.

6. A kit of parts suitable for performing a method for identifying and/or characterising a target cell in, and/or isolating a target cell from, a test sample which may contain said target cell, comprising
1) a target cell selective reagent suitable for interacting with a characterising component of the target cell and
2) a positive control cell, wherein the positive control cell comprises said characterising component and is not identical to the target cell.

7. The kit of parts according to claim 6 wherein the kit further comprises one or more further reagents capable of interacting with one or more further components of the target cell and wherein the positive control cell further comprises said further component(s).

8. The kit of parts according to claim 6 or 7 wherein the kit further comprises a negative control cell as defined in claim 5.

9. The method, use or kit of parts of any of the preceding claims wherein the characterising component is a cell surface component.

10. The method, use or kit of parts of any of claims 1 to 9 wherein the characterising component is a polynucleotide.

11. The method, use or kit of parts of claim 4, 5, 7, 8, 9 or 10 (when dependent on claims 4, 5 or 7) wherein a said further component is a polynucleotide.

12. The method, use or kit of parts of claim 4, 5, 7, 8, 9 or 10 (when dependent on claims 4, 5 or 7) or claim 11 wherein a said further component is a polypeptide.

13. The method, use or kit of parts of any of the preceding claims wherein the target cell is a fetal or embryonic red blood cell.

14. The method, use or kit of parts of claim 13 wherein the characterising component is an adult liver component.

15. The method, use or kit of parts of claim 14 wherein the characterising component is GLUT2, a P-glycoprotein, a MDRP, a MRP, γ-glutamyl transpeptidase, a lipoprotein receptor, an alkaline phosphatase, a bile salt transporter, a bile acid transporter, a hormone receptor, a MOAT, a bilirubin transporter or a bilirubin conjugate transporter.

16. The method, use or kit of parts of any one of claims 13 to 15 wherein a further component is glucose-6-phosphatase or UDP glucuronosyltransferase.

17. The method, use or kit of parts of any one of claims 13 to 16 or of claim 4, 5, 7, 8, 9 or 10 (when dependent on claims 4, 5 or 7) wherein a further component is a component linked with a genetic disease or condition (including sex) or fetal abnormality.

18. The method, use or kit of parts of claim 17 wherein the component linked with a genetic disease or condition (including sex) or fetal abnormality is a polynucleotide.

19. The method, use or kit of parts of any of the preceding claims wherein the positive control cell is recombinant.

20. A recombinant mammalian cell comprising two or more human adult liver components and optionally a polynucleotide sequence associated with, or associated with the absence of, a human disease, condition, or trait.

21. A method of testing a procedure for identifying and/or characterising a target cell in, and/or isolating a target cell from, a test sample which may contain said target cell, comprising the steps of
1) providing a target cell selective reagent which interacts with a characterising component derivable from the target cell,
2) providing a positive control sample comprising a positive control cell, wherein the positive control cell comprises said characterising component, is recombinant, and is not identical to the target cell,
3) identifying and/or characterising the positive control cell in, and/or isolating the positive control cell from, the positive control sample by performing on the sample a process involving interaction of the test cell selective reagent with the characterising component of the positive control cell,
wherein the method for identifying, isolating and/or characterising the target cell is considered to be satisfactory if the positive control cell is identified, isolated and/or characterised satisfactorily in step 3.

22. The method, use or kit of parts of any one of claims 1 to 12 wherein the target cell is a bacterial cell.

23. The method, use or kit of parts of any one of claims 1 to 12 wherein the target cell is a cancer cell.

24. A solid support suitable for use in a method for identifying and/or characterising a target cell in, and/or isolating a target cell from, a test sample, on which is immobilised 1) a positive control cell, wherein the positive control cell comprises a characterising component and 2) a negative control cell, wherein the negative control cell does not comprise the characterising component.

25. A solid support according to claim 24 wherein the positive control cell is a recombinant cell as defined in claim 20.
